(19) United States
(12) Patent Application Publication
(10) Pub. No.: US 2018/0050989 A1
(43) Pub. Date: Feb. 22, 2018

(54) ALDEHYDE TRAPPING COMPOUNDS AND USES THEREOF

(71) Applicant: Aldeyra Therapeutics, Inc., Lexington, MA (US)

(72) Inventors: Stephen Gitu Machatha, Burlington, MA (US); Scott Young, E. Falmouth, MA (US)

(21) Appl. No.: 15/682,955

(22) Filed: Aug. 22, 2017

Related U.S. Application Data
(60) Provisional application No. 62/378,065, filed on Aug. 22, 2016.

(51) Int. Cl. C07D 215/38; A61K 31/47; A61K 9/00; C07D 401/04; A61K 31/4709

(52) U.S. Cl. CPC C07D 215/38; A61K 31/47; A61K 31/4709; C07D 401/04; A61K 9/0014

(57) ABSTRACT

The present invention provides compounds and methods for the treatment, prevention, and/or reduction of a risk of a disease, disorder, or condition in which aldehyde toxicity is implicated in the pathogenesis, including ocular disorders, skin disorders, conditions associated with injurious effects from blister agents, and autoimmune, inflammatory, neurological and cardiovascular diseases by the use of a primary amine to scavenge toxic aldehydes, such as MDA and HNE.

### CROSS-REFERENCE TO RELATED APPLICATIONS

This application claims the benefit of U.S. Provisional Application No. 62/378,065, filed on Aug. 22, 2016, the entirety of which is hereby incorporated by reference.

### BACKGROUND OF THE INVENTION

Metabolic and inflammatory processes in cells generate toxic aldehydes, such as malondialdehyde (MDA) and 4-hydroxy-2-nonenal (HNE or 4-HNE). These aldehydes are highly reactive to proteins, carbohydrates, lipids and DNA, leading to chemically modified biological molecules, activation of inflammatory mediators such as NF-kappaB, and damage in diverse organs. For example, retinaldehyde can react with phosphatidylethanolamine (PE) to form a highly toxic compound called A2E, which is a component of lipofuscin believed to be involved in the development and progression of Age-Related Macular Degeneration (AMD). Many bodily defense mechanisms function to remove or lower the levels of toxic aldehydes. Novel small molecule therapeutics can be used to scavenge “escaped” retinaldehyde in the retina, thus reducing A2E formation and lessening the risk of AMD (Jordan et al. (2006)).

Aldehydes are implicated in diverse pathological conditions such as dry eye, cataracts, keratoconus, Fuch's endothelial dystrophy in the cornea, uveitis, allergic conjunctivitis, succinic semialdehyde dehydrogenase deficiency (SSADHD), pyridoxine-dependent epilepsy (ALDH7A1 mutation), ocular cicatricial pemphigoid, conditions associated with photorefractive keratectomy (PRK) healing or other corneal healing, conditions associated with tear lipid degradation or lacrimal gland dysfunction, inflammatory ocular conditions such as ocular rosacea (with or without meibomian gland dysfunction), and non-ocular disorders or conditions such as skin cancer, psoriasis, contact dermatitis, atopic dermatitis, acne vulgaris, Sjögren-Larsson Syndrome, ischemic-reperfusion injury, inflammation, diabetes, neurodegeneration (e.g., Parkinson's disease), scleroderma, amyotrophic lateral sclerosis, autoimmune disorders (e.g., lupus), cardiovascular disorders (e.g., atherosclerosis), and conditions associated with the injurious effects of blister agents (Negre-Salvagre et al. (2008), Nakamura et al. (2007), Batista et al. (2012), Kenney et al. (2003), Int J Dermatol 43: 494 (2004), Invest Ophthalmol Vis Sci 48: 1552 (2007), Graefe's Clin Exp Ophthalmol 233: 694 (1994), Molecular Vision 18: 194 (2012)). Reducing or eliminating aldehydes should thus ameliorate the symptoms and slow the progression of these pathological conditions.

MDA, HNE and other toxic aldehydes are generated by a myriad of metabolic mechanisms involving: fatty alcohols, sphingolipids, glycolipids, phytol, fatty acids, arachadonic acid metabolism (Rizzo (2007)), polyamine metabolism (Wood et al. (2006)), lipid peroxidation, oxidative metabolism (Buddi et al. (2002), Zhou et al. (2005)), and glucose metabolism (Pozzi et al. (2009)). Aldehydes can cross link with primary amino groups and other chemical moieties on proteins, phospholipids, carbohydrates, and DNA, leading in many cases to toxic consequences, such as mutagenesis and carcinogenesis (Marnett (2002)). MDA is associated with diseased corneas, keratoconus, bullous and other keratopathy, and Fuch's endothelial dystrophy corneas (Buddi et al. (2002)). Also, skin disorders, e.g., ichthyosis associated with Sjögren-Larsson Syndrome, are likely connected with the accumulation of fatty aldehydes such as octadecanal and hexadecanal (Rizzo et al. (2010)). Further, increased lipid peroxidation and resultant aldehyde generation are associated with the toxic effects of blister agents (Sciuto et al. (2004) and Pal et al. (2009)).

There has been no suggestion in the art for treating the various conditions associated with toxic aldehydes by the administration of small molecule therapeutics acting as a scavenger for aldehydes, such as MDA and/or HNE. Thus, there is a need for treating, preventing, and/or reducing a risk of a disease or disorder in which aldehyde toxicity is implicated in the pathogenesis. The present invention addresses such a need.

Accordingly, there remains a need for treating, preventing, and/or reducing a risk of a disease, disorder, or condition in which aldehyde toxicity is implicated in the pathogenesis.

### SUMMARY OF THE INVENTION

It has now been found that compounds of the present invention, and compositions thereof, are useful for treating, preventing, and/or reducing a risk of a disease, disorder, or condition in which aldehyde toxicity is implicated in the pathogenesis. In one aspect of the present invention, such compounds have general formula I:

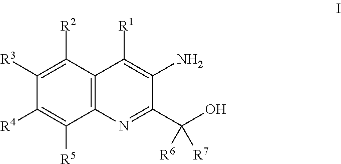

or a pharmaceutically acceptable salt thereof, wherein each of R¹, R², R³, R⁴, R⁵, R⁶, and R⁷ is as defined herein.

Compounds of the present invention, and pharmaceutically acceptable compositions thereof, are useful for treating a variety of diseases, disorders or conditions, associated with toxic aldehydes. Such diseases, disorders, or conditions include those described herein.

Compounds provided by this invention are also useful for the study of certain aldehydes in biology and pathological phenomena.

### BRIEF DESCRIPTION OF THE DRAWINGS

FIG. 1 shows rates of formation of aldehyde adducts over a 23 h time period for NS2 and exemplary compounds of the present invention.

FIG. 2 shows consumption of 4-HNE over time (23-hour formation period) for NS2 and exemplary compounds of the present invention.

FIG. 3 shows rates of formation of aldehyde adducts over a 1 week time period for NS2 and exemplary compounds of the present invention to measure whether compounds reached equilibrium. During this time period 3 of the 5 samples reached equilibrium.

FIG. 4 shows consumption of 4-HNE over a 1 week time period for NS2 and exemplary compounds of the present invention to measure whether compounds reached equilibrium during this time period.

FIG. 5 shows the effect of NS2 on measured GABA and GHB content in brain slices and associated incubation fluid from B6.129-Aldh5a1^{tm1Kmg}/J (SSADH-deficient) mice.

FIG. 6 shows effects of compound I-1 on levels of GABA and GHB in brain slices and associated incubation fluid from B6.129-Aldh5a1^{tm1Kmg}/J (SSADH-deficient) mice.

FIG. 7 shows results for an assay measuring formation of the 4-HNE adduct with NS2. The assay was performed twice, with the measurements on different days. NS2 formed the corresponding adduct with 4-HNE. The two results were similar to each other, and were close enough to be within the measurement error for the HPLC instrument.

FIG. 8 shows results for an assay measuring formation of the 4-HNE adduct with I-1. The assay was performed twice, with the measurements on different days. I-1 formed the corresponding adduct with 4-HNE. The two results were similar to each other, and were close enough to be within the measurement error for the HPLC instrument.

### DETAILED DESCRIPTION OF THE INVENTION

### 1. General Description of Certain Aspects of the Invention

In certain embodiments, the present invention provides compounds, compositions, and methods for treatment, prevention, and/or reduction of a risk of diseases, disorders, or conditions in which aldehyde toxicity is implicated in the pathogenesis. In some embodiments, such compounds include those of the formulae described herein, or a pharmaceutically acceptable salt thereof, wherein each variable is as defined herein and described in embodiments. In some embodiments, a disclosed compound contains an amino functionality and a carbinol functionality (such as a propan-2-ol group) that are believed to be capable of scavenging or trapping aldehydes by formation of an adduct. Such compounds have the structure of formula I:

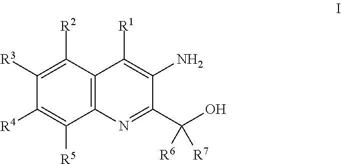

or a pharmaceutically acceptable salt thereof, wherein:

R¹ is H, D, or halogen;

R² is H, D, or halogen;

R³ is H, D, Br, or I;

R⁴ is H, D, or halogen;

R⁵ is H, D, or halogen;

R⁶ is C₁₋₄ aliphatic optionally substituted with 1, 2, or 3 deuterium or halogen atoms; and

R⁷ is C₁₋₄ aliphatic optionally substituted with 1, 2, or 3 deuterium or halogen atoms.

### 2. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

The “retina” is a region of the central nervous system with approximately 150 million neurons. It is located at the back of the eye where it rests upon a specialized epithelial tissue called retinal pigment epithelium (RPE). The retina initiates the first stage of visual processing by transducing visual stimuli in specialized neurons called “photoreceptors”. Their synaptic outputs are processed by elaborate neural networks in the retina and are then transmitted to the brain. The retina has evolved two specialized classes of photoreceptors to operate under a wide range of light conditions. “Rod” photoreceptors transduce visual images under low light conditions and mediate achromatic vision. “Cone” photoreceptors transduce visual images in dim to bright light conditions and mediate both color vision and high acuity vision.

Every photoreceptor is compartmentalized into two regions called the “outer” and “inner” segment. The inner segment is the neuronal cell body containing the cell nucleus. The inner segment survives for a lifetime in the absence of retinal disease. The outer segment is the region where the light sensitive visual pigment molecules are concentrated in a dense array of stacked membrane structures. Part of the outer segment is routinely shed and regrown in a diurnal process called outer segment renewal. Shed outer segments are ingested and metabolized by RPE cells.

The “macula” is the central region of the retina which contains the fovea where visual images are processed by long slender cones in high spatial detail (“visual acuity”). “Macular degeneration” is a form of retinal neurodegeneration which attacks the macula and destroys high acuity vision in the center of the visual field. Age-Related Macular Degeneration (AMD) begins in a “dry form” characterized by residual lysosomal granules called lipofuscin in RPE cells, and by extracellular deposits called “drusen”. Drusen contain cellular waste products excreted by RPE cells. “Lipofuscin” and drusen can be detected clinically by ophthalmologists and quantified using fluorescence techniques. They can be the first clinical signs of macular degeneration.

Lipfuscin contains aggregations of A2E. Lipofuscin accumulates in RPE cells and poisons them by multiple known mechanisms. As RPE cells become poisoned, their biochemical activities decline and photoreceptors begin to degenerate. Extracellular drusen may further compromise RPE cells by interfering with their supply of vascular nutrients. Drusen also trigger inflammatory processes, which lead to choroidal neovascular invasions of the macula in one patient in ten who progresses to wet form AMD. Both the dry form and wet form progress to blindness.

“ERG” is an acronym for electroretinogram, which is the measurement of the electric field potential emitted by retinal neurons during their response to an experimentally defined light stimulus. ERG is a non-invasive measurement which can be performed on either living subjects (human or animal) or a hemisected eye in solution that has been removed surgically from a living animal.

As used herein, the term “RAL” means retinaldehyde. The term “RAL-trap” means a therapeutic compound that binds free RAL and thereby prevents the RAL from Schiff base condensation with membrane phosphatidylethanolamine (PE). “Free RAL” is defined as RAL that is not bound to a visual cycle protein. The terms “trans-RAL” and “all-trans-RAL” are used interchangeably and mean all trans-retinaldehyde.

A2E is a reaction by-product of a complex biochemical pathway called the “visual cycle” which operates collaboratively in both RPE cells and photoreceptor outer segments. The visual cycle recycles a photoreactive aldehyde chromophore called “retinaldehyde” which is derived from vitamin A and is essential for vision. In simplified terms, the visual cycle has four principal steps: 1) it converts vitamin A in the RPE into an aldehyde chromophore with one photoreactive strained double bond (11-cis-RAL); 2) it transports 11-cis-RAL to the retina where it binds to a specialized photoreceptor protein called opsin; 3) light photoisomerizes bound 11-cis-RAL to trans-RAL, which initiates the release of bound RAL from the opsin binding site; and 4) it converts trans-RAL (an aldehyde) to vitamin A (an alcohol) and transports vitamin A back to the RPE where the cycle begins again.

The aldehyde group of RAL helps bind the molecule to opsin by forming a reversible chemical bond to an amino acid sidechain in the opsin binding site. While the aldehyde group on RAL is essential for anchoring the molecule to the opsin binding site, it is otherwise hazardous because of its propensity to form Schiff bases with other biological amines. The first three reactions take place in photoreceptor outer segments and produce an intermediary product called A2PE. Once formed, A2PE partitions into the lipid phase and accumulates in photoreceptor outer segment membranes.

As described above, macular degeneration and other forms of retinal disease whose etiology involves the accumulation of A2E and/or lipofuscin may be treated or prevented by lowering the amount of A2E formed. Compounds useful for doing so include RAL-traps. RAL-traps lower the amount of A2E formed, for example by forming a covalent bond with RAL that has escaped sequestering. RAL that has reacted with a RAL-trap compound is thereby unavailable to react with phosphatidylethanolamine.

The present invention is also directed to the use of a compound described herein in the manufacture of a medicament for the treatment, prevention, and/or reduction of a risk of a disease, disorder, or condition in which aldehyde toxicity is implicated in the pathogenesis. More specifically, this aspect of the invention is directed to the use of a compound described herein in the manufacture of a medicament for the treatment, prevention, and/or reduction of a risk of (1) an ocular disease, disorder, or condition, including, but not limited to, a corneal disease (e.g., dry eye syndrome, cataracts, keratoconus, bullous and other keratopathy, and Fuch's endothelial dystrophy), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions associated with high aldehyde levels as a result of inflammation (e.g., uveitis, scleritis, ocular Stevens-Johnson Syndrome, and ocular rosacea (with or without meibomian gland dysfunction)), (2) a skin disorder or condition or a cosmetic indication. For example, the disease, disorder, or condition includes, but is not limited to, psoriasis, topical (discoid) lupus, contact dermatitis, atopic dermatitis, allergic dermatitis, radiation dermatitis, acne vulgaris, Sjögren-Larsson Syndrome and/or associated ichthyoses, solar elastosis/wrinkles, skin tone firmness, puffiness, eczema, smoke or irritant induced skin changes, dermal incision, and a skin condition associated with a burn or wound, (3) a condition associated with the toxic effects of blister agents or burns from alkali agents, or (4) an autoimmune, immune-mediated, inflammatory, cardiovascular, or neurological disease such as lupus, scleroderma, asthma, chronic obstructive pulmonary disease (COPD), rheumatoid arthritis, inflammatory bowel disease, sepsis, atherosclerosis, ischemic-reperfusion injury, Parkinson's disease, Alzheimer's disease, multiple sclerosis, amyotrophic lateral sclerosis, diabetes, metabolic syndrome, a fibrotic disease, neurogical and/or motor effects of SLS, SSADHD, and pyridoxine-dependent epilepsy.

The present invention is also directed to the use of a compound described herein in treating, preventing, and/or reducing a risk of a disease, disorder, or condition in which aldehyde toxicity is implicated in the pathogenesis. More specifically, this aspect of the invention is directed to the use of a compound described herein in treating, preventing, and/or reducing a risk of (1) an ocular disease, disorder, or condition, including, but not limited to, a corneal disease (e.g., dry eye syndrome, cataracts, keratoconus, bullous and other keratopathy, and Fuch's endothelial dystrophy), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions associated with high aldehyde levels as a result of inflammation (e.g., uveitis, scleritis, ocular Stevens-Johnson Syndrome, and ocular rosacea (with or without meibomian gland dysfunction)), (2) a skin disorder or condition or a cosmetic indication, for example, psoriasis, topical (discoid) lupus, contact dermatitis, atopic dermatitis, allergic dermatitis, radiation dermatitis, acne vulgaris, Sjögren-Larsson Syndrome and/or associated ichthyoses, solar elastosis/wrinkles, skin tone firmness, puffiness, eczema, smoke or irritant induced skin changes, dermal incision, and a skin condition associated burn and wound, (3) a condition associated with the toxic effects of blister agents or burns from alkali agents, or (4) an autoimmune, immune-mediated, inflammatory, cardiovascular, or neurological disease such as lupus, scleroderma, asthma, chronic obstructive pulmonary disease (COPD), rheumatoid arthritis, inflammatory bowel disease, sepsis, atherosclerosis, ischemic-reperfusion injury, Parkinson's disease, Alzheimer's disease, multiple sclerosis, amyotrophic lateral sclerosis, diabetes, metabolic syndrome, a fibrotic disease, neurogical and/or motor effects of SLS, SSADHD, and pyridoxine-dependent epilepsy.

The compounds described herein can also be administered topically, such as directly to the eye, e.g., as an eye-drop or ophthalmic ointment. Eye drops typically comprise an effective amount of at least one compound described herein and a carrier capable of being safely applied to an eye. For example, the eye drops are in the form of an isotonic solution, and the pH of the solution is adjusted so that there is no irritation of the eye. In many instances, the epithelial barrier interferes with penetration of molecules into the eye. Thus, most currently used ophthalmic drugs are supplemented with some form of penetration enhancer. These penetration enhancers work by loosening the tight junctions of the most superior epithelial cells (Burstein, Trans Ophthalmol Soc UK 104: 402 (1985); Ashton et al., J Pharmacol Exp Ther 259: 719 (1991); Green et al., Am J Ophthalmol 72: 897 (1971)). The most commonly used penetration enhancer is benzalkonium chloride (Tang et al., J Pharm Sci 83: 85 (1994); Burstein et al, Invest Ophthalmol Vis Sci 19: 308 (1980)), which also works as preservative against microbial contamination.

Topical administration may be in the form of a cream, suspension, emulsion, ointment, drops, oil, lotion, patch, tape, inhalant, spray, or controlled release topical formulations including gels, films, patches, and adhesives. Intra-ocular administration may take the form of subconjunctival, subtenon's capsule, retrobulbar or intravitreal injections, depots or implants. Compounds administered by these routes may be in solution or suspension form. Administration of compounds by depot injection may contain pharmaceutically acceptable carriers or excipients; these may be natural or synthetic and may be biodegradable or non-biodegradable and facilitate drug release in a controlled manner. Implants used for controlled release of compound may be composed of natural or synthetic, biodegradable or non-biodegradable materials. The carrier is acceptable in that it is compatible with the other components of the composition and is not injurious to the patient. Some examples of carriers include (1) sugars such as lactose glucose and sucrose, (2) starches such as corn starch and potato starch, (3) cellulose and (4) cyclodextrins. A useful topical formulation is described in PCT publication WO 2011/072141, the contents of which are herein incorporated by reference.

Formulations for topical administration to the skin can include, for example, ointments, creams, gels and pastes comprising the primary amine compound in a pharmaceutical acceptable carrier. The formulation of the primary amine compound for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

The formulations for topical administration may contain the compound used in the present application at a concentration in the range of 0.001-10%, 0.05-10%, 0.1-10%, 0.2-10%, 0.5-10%, 1-10%, 2-10%, 3-10%, 4-10%, 5-10%, or 7-10% (weight/volume), or in the range of 0.001-2.0%, 0.001-1.5%, or 0.001-1.0%, (weight/volume), or in the range of 0.05-2.0%, 0.05-1.5%, or 0.05-1.0%, (weight/volume), or in the range of 0.1-5.0%, 0.1-2.0%, 0.1-1.5%, or 0.1-1.0% (weight/volume), or in the range of 0.5-5.0%, 0.5-2.0%, 0.5-1.5%, or 0.5-1.0% (weight/volume), or in the range of 1-5.0%, 1-2.0%, or 1-1.5% (weight/volume). The formulations for topical administration may also contain the compound used in the present application at a concentration in the range of 0.001-2.5%, 0.01-2.5%, 0.05-2.0%, 0.1-2.0%, 0.2-2.0%, 0.5-2.0%, or 1-2.0% (weight/weight), or in the range of 0.001-2.0%, 0.001-1.5%, 0.001-1.0%, or 0.001-5% (weight/weight).

In an eye drop formulation the composition may contain the active compound at a concentration of 0.01-20%, 0.02-15%, 0.04-10%, 0.06-5%, 0.08-1%, or 0.09-0.5% (weight/volume) with or without pH and/or osmotic adjustment to the solution. More particularly, the eye drop formulation may contain a compound described herein at a concentration of 0.09-0.5% (weight/volume), such as 0.1%.

In one exemplification, the pharmaceutical compositions encompass a composition made by admixing a therapeutically effective amount of a compound described herein with an oligomeric or a polymeric carrier such as a cyclodextrin, or chemically modified cyclodextrin, including trimethyl-β-cyclodextrin, 2-hydroxyethyl-β-cyclodextrin, 2-hydroxypropyl-β-cyclodextrin, 3-hydroxypropyl-β-cyclodextrin, and β-cyclodextrin sulfobutylether sodium salt (or potassium salt). Exemplifying an oligomeric or a polymeric carrier is β-cyclodextrin sulfobutylether sodium salt. The amount of β-cyclodextrin sulfobutylether sodium salt in the composition may range from about 0.01% to 30% weight/volume. In one illustration, the concentration of β-cyclodextrin sulfobutylether sodium salt is 5-25% weight/volume. Further illustrating the concentration of β-cyclodextrin sulfobutylether sodium salt is 6-20% weight/volume. In one exemplification, the concentration of β-cyclodextrin sulfobutylether is 6-12% weight/volume. Further exemplifying the concentration of β-cyclodextrin sulfobutylether is 9-10% weight/volume, including 9.5% weight/volume. The amount of the compound described herein in the composition may range 0.01-20%, 0.02-15%, 0.04-10%, 0.06-5%, 0.08-1%, or 0.09-0.5% (weight/volume). More particularly, the composition may contain a compound described herein at a concentration of 0.09-0.5% (weight/volume), such as 0.1%.

The compounds described herein may be administered orally and as such the pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

For oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, croscarmellose or its sodium salt, and the like. Diluents, include, e.g., lactose, dextrose, sucrose, mannitol, sorbitol, cellulose and/or glycine.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A therapeutically effective dose, of a compound described herein in an oral formulation, may vary from 0.01 mg/kg to 50 mg/kg patient body weight per day, more particularly 0.01 to 10 mg/kg, which can be administered in single or multiple doses per day. For oral administration, the drug can be delivered in the form of tablets or capsules containing 1 mg to 500 mg of the active ingredient specifically, 1 mg, 5 mg, 10 mg, 20 mg, 50 mg, 100 mg, 250 mg, and 500 mg, or in the forms of tables or capsules containing at least 1%, 2%, 5%, 10%, 15%, 20%, 25%, 30%, 40%, 50% (w/w) of the active ingredient. For example, the capsules may contain 50 mg of the active ingredient, or 5-10% (w/w) of the active ingredient. For example, the tablets may contain 100 mg of the active ingredient, or 20-50% (w/w) of the active ingredient. For example, the tablet may contain, in addition to the active ingredient, a disintegrant or emollient (e.g., croscarmellose or its sodium salt and methyl cellulose), a diluent (e.g., microcrystalline cellulose), and a lubricant (e.g., sodium stearate and magnesium stearate). The drug can be administered on a daily basis either once, twice or more per day.

For administration by inhalation, the compounds are delivered in the form of an aerosol spray from pressured container or dispenser, which contains a suitable propellant, e.g., a gas such as carbon dioxide, or a nebulizer.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Parenteral formulations comprising a compound described herein can be prepared in aqueous isotonic solutions or suspensions, and suppositories are advantageously prepared from fatty emulsions or suspensions. The formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional methods, and may contain about 0.1 to 75%, preferably about 1 to 50%, of a compound described herein.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

### 3. Description of Exemplary Compounds

It has now been found that compounds of the present invention, and compositions thereof, are useful for treating, preventing, and/or reducing a risk of a disease, disorder, or condition in which aldehyde toxicity is implicated in the pathogenesis.

According to one aspect, the present invention provides a compound of formula I:

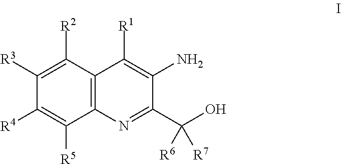

or a pharmaceutically acceptable salt thereof, wherein:

R¹ is H, D, or halogen;

R² is H, D, or halogen;

R³ is H, D, Br, or I;

R⁴ is H, D, or halogen;

R⁵ is H, D, or halogen;

R⁶ is C₁₋₄ aliphatic optionally substituted with 1, 2, or 3 deuterium or halogen atoms; and

R⁷ is C₁₋₄ aliphatic optionally substituted with 1, 2, or 3 deuterium or halogen atoms.

As defined generally above, R¹ is H, D, or halogen.

In some embodiments, R¹ is H. In some embodiments, R¹ is D. In some embodiments, R¹ is halogen. In some embodiments, R¹ is Cl. In some embodiments, R¹ is Br.

As defined generally above, R² is H, D, or halogen.

In some embodiments, R² is H. In some embodiments, R² is D. In some embodiments, R² is halogen. In some embodiments, R² is Cl. In some embodiments, R² is Br.

As defined generally above, R³ is H, D, Br, or I.

In some embodiments, R³ is H. In some embodiments, R³ is D. In some embodiments, R³ is Br. In some embodiments, R³ is I.

As defined generally above, R⁴ is H, D, or halogen.

In some embodiments, R⁴ is H. In some embodiments, R⁴ is D. In some embodiments, R⁴ is halogen. In some embodiments, R⁴ is Cl. In some embodiments, R⁴ is Br.

As defined generally above, R⁵ is H, D, or halogen.

In some embodiments, R⁵ is H. In some embodiments, R⁵ is D. In some embodiments, R⁵ is halogen. In some embodiments, R⁵ is Cl. In some embodiments, R⁵ is Br.

As defined generally above, R⁶ is C₁₋₄ aliphatic optionally substituted with 1, 2, or 3 deuterium or halogen atoms.

In some embodiments, R⁶ is C₁₋₄ aliphatic substituted with 1, 2, or 3 deuterium or halogen atoms. In some embodiments, R⁶ is C₁₋₄ aliphatic. In some embodiments, R⁶ is C₁₋₄ alkyl. In some embodiments, R⁶ is methyl, ethyl, n-propyl, or isopropyl. In some embodiments, R⁶ is methyl.

As defined generally above, R⁷ is C₁₋₄ aliphatic optionally substituted with 1, 2, or 3 deuterium or halogen atoms.

In some embodiments, R⁷ is C₁₋₄ aliphatic substituted with 1, 2, or 3 deuterium or halogen atoms. In some embodiments, R⁷ is C₁₋₄ aliphatic. In some embodiments, R⁷ is C₁₋₄ alkyl. In some embodiments, R⁷ is C₁₋₄ alkyl optionally substituted with 1, 2, or 3 fluorine atoms. In some embodiments, R⁷ is methyl, ethyl, n-propyl, or isopropyl. In some embodiments, R⁷ is methyl.

In some embodiments, R⁶ and R⁷ are methyl or ethyl. In some embodiments, R⁶ and R⁷ are methyl.

In another aspect, the present invention provides a compound of formula I-a:

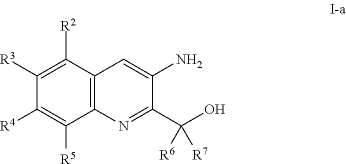

or a pharmaceutically acceptable salt thereof, wherein:
each of R², R³, R⁴, R⁵, R⁶, and R⁷ is as defined is as defined above and described in embodiments herein, both singly and in combination.

In another aspect, the present invention provides a compound of formula I-b:

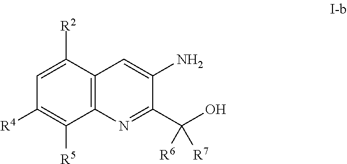

or a pharmaceutically acceptable salt thereof, wherein:
each of R², R⁴, R⁵, R⁶, and R⁷ is as defined is as defined above and described in embodiments herein, both singly and in combination.

In another aspect, the present invention provides a compound of formulae I-c, I-d, I-e, or I-f:

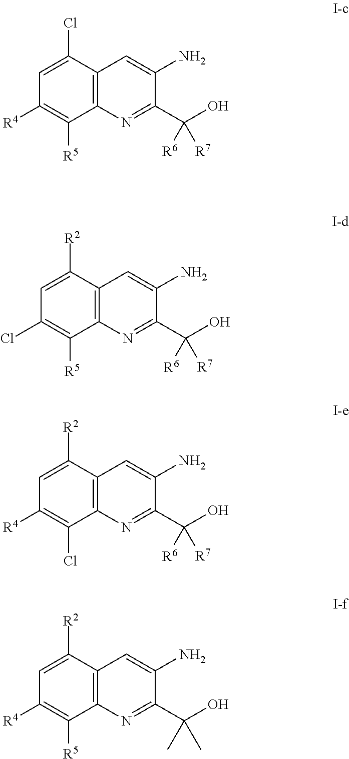

or a pharmaceutically acceptable salt thereof, wherein:
each of R², R⁴, R⁵, R⁶, and R⁷ is as defined is as defined above and described in embodiments herein, both singly and in combination.

In another aspect, the present invention provides a compound of formulae I-g, I-h, I-i, or I-j:

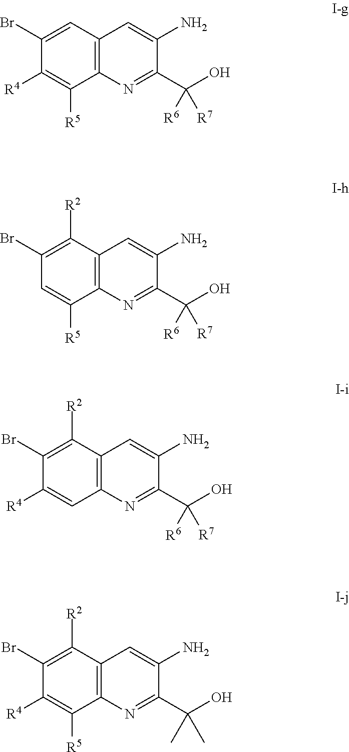

or a pharmaceutically acceptable salt thereof, wherein:
each of R², R⁴, R⁵, R⁶, and R⁷ is as defined is as defined above and described in embodiments herein, both singly and in combination.

In another aspect, the present invention provides a compound of formula I-k or I-l:

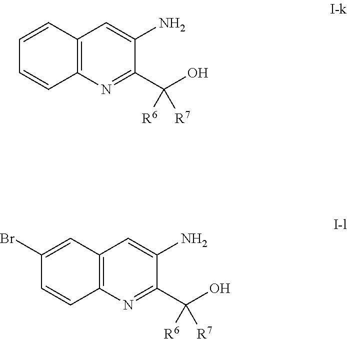

or a pharmaceutically acceptable salt thereof, wherein:
each of R⁶ and R⁷ is as defined is as defined above and described in embodiments herein, both singly and in combination.

In another aspect, the present invention provides a composition comprising a compound of formula II:

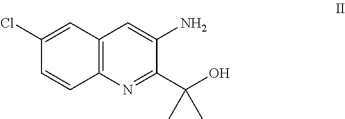 [SMILES: CC(C)(O)c1nc2ccc(Cl)cc2cc1N]

or a pharmaceutically acceptable salt thereof, and at least one compound of formula I:

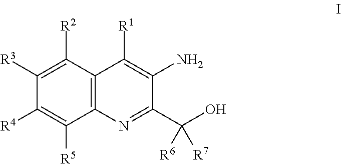

or a pharmaceutically acceptable salt thereof, wherein:

R¹ is H, D, or halogen;

R² is H, D, or halogen;

R³ is H, D, Br, or I;

R⁴ is H, D, or halogen;

R⁵ is H, D, or halogen;

R⁶ is C₁₋₄ aliphatic optionally substituted with 1, 2, or 3 deuterium or halogen atoms; and

R⁷ is C₁₋₄ aliphatic optionally substituted with 1, 2, or 3 deuterium or halogen atoms.

In some embodiments, the present invention provides a composition comprising a compound of formula II, or a pharmaceutically acceptable salt thereof, and at least one compound according to formulae I-a, I-b, I-c, I-d, I-e, I-f, I-g, I-h, I-i, I-j, I-k, or I-l; or a pharmaceutically acceptable salt thereof.

In another aspect, the present invention provides a composition comprising a compound of formula II:

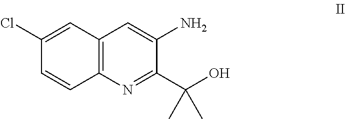 [SMILES: CC(C)(O)c1nc2ccc(Cl)cc2cc1N]

or a pharmaceutically acceptable salt thereof, and a compound selected from the following, or a pharmaceutically acceptable salt thereof:

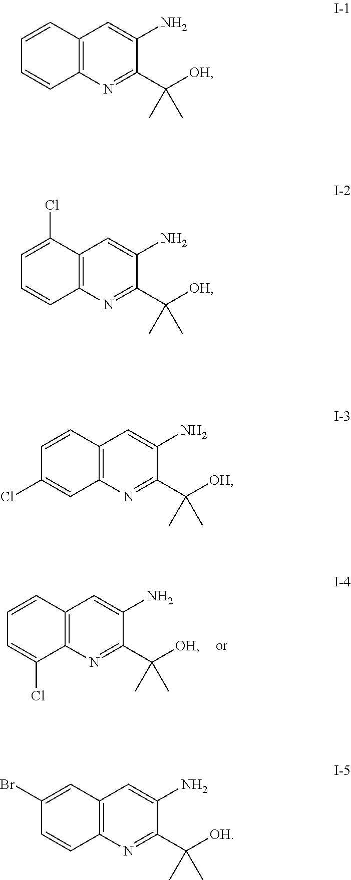 [SMILES: CC(C)(O)c1nc2ccccc2cc1N ; CC(C)(O)c1nc2cccc(Cl)c2cc1N ; CC(C)(O)c1nc2cc(Cl)ccc2cc1N ; CC(C)(O)c1nc2c(Cl)cccc2cc1N ; CC(C)(O)c1nc2ccc(Br)cc2cc1N]

In some embodiments, the composition comprises a compound of formula II, or a pharmaceutically acceptable salt thereof, and one additional compound selected from I-1, I-2, I-3, I-4, or I-5; or a pharmaceutically acceptable salt thereof.

In some embodiments, the composition comprises a compound of formula II, or a pharmaceutically acceptable salt thereof, and two additional compounds selected from I-1, I-2, I-3, I-4, or I-5; or a pharmaceutically acceptable salt thereof.

In some embodiments, the composition comprises a compound of formula II, or a pharmaceutically acceptable salt thereof, and three additional compounds selected from I-1, I-2, I-3, I-4, or I-5; or a pharmaceutically acceptable salt thereof.

In some embodiments, the composition comprises a compound of formula II, or a pharmaceutically acceptable salt thereof, and four additional compounds selected from I-1, I-2, I-3, I-4, or I-5; or a pharmaceutically acceptable salt thereof.

In some embodiments, the composition comprises a compound of formula II, or a pharmaceutically acceptable salt thereof, and one additional compound selected from I-2, I-3, or I-4; or a pharmaceutically acceptable salt thereof.

In some embodiments, the composition comprises a compound of formula II, or a pharmaceutically acceptable salt thereof, and two additional compounds selected from I-2, I-3, or I-4; or a pharmaceutically acceptable salt thereof. In some embodiments, the composition comprises I-2, I-3, and I-4; or a pharmaceutically acceptable salt thereof.

In some embodiments, the composition comprises a compound of formula II, or a pharmaceutically acceptable salt thereof, and I-1; or a pharmaceutically acceptable salt thereof.

In some embodiments, the composition comprises a compound of formula II, or a pharmaceutically acceptable salt thereof, and I-2; or a pharmaceutically acceptable salt thereof.

In some embodiments, the composition comprises a compound of formula II, or a pharmaceutically acceptable salt thereof, and I-3; or a pharmaceutically acceptable salt thereof.

In some embodiments, the composition comprises a compound of formula II, or a pharmaceutically acceptable salt thereof, and I-4; or a pharmaceutically acceptable salt thereof.

In some embodiments, the composition comprises a compound of formula II, or a pharmaceutically acceptable salt thereof, and I-5; or a pharmaceutically acceptable salt thereof.

In another aspect, the present invention provides a compound of formula I selected from these depicted in Table 1, below.

TABLE 1
Representative Compounds of Formula I

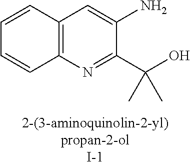

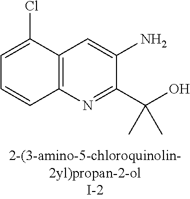 [SMILES: CC(C)(O)c1nc2cccc(Cl)c2cc1N]

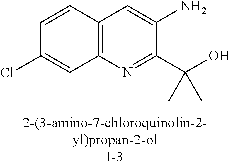 [SMILES: CC(C)(O)c1nc2cc(Cl)ccc2cc1N]

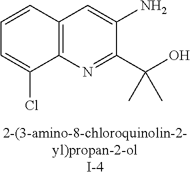 [SMILES: CC(C)(O)c1nc2c(Cl)cccc2cc1N]

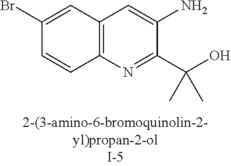 [SMILES: CC(C)(O)c1nc2ccc(Br)cc2cc1N]

In some embodiments, the present invention provides a compound depicted in Table 1, above, or a pharmaceutically acceptable salt thereof.

In certain embodiments, the present invention provides any compound described above and herein, or a pharmaceutically acceptable salt thereof.

In other embodiments, the composition contains a compound of any one of formulae I, I-a, I-b, I-c, I-d, I-e, I-f, I-g, I-h, I-i, I-j, I-k, or I-l, or a pharmaceutically acceptable salt thereof, in an amount of at least about 97, 97.5, 98, 98.5, 99.0, 99.5, 99.8, 99.9, 99.95, or 99.999 weight percent where the percentages are based on the free base of said compound and the total weight of the composition. In other embodiments, the composition contains no more than about 2.0 area percent HPLC of total organic impurities or, in other embodiments, no more than about 1.5, 1.25, 1, 0.75, 0.5, 0.25, 0.2, 0.1, 0.01, 0.005, or 0.001 area percent HPLC total organic impurities relative to the total area of the HPLC chromatogram.

In other embodiments, a composition is provided comprising a compound of formula II or a pharmaceutically acceptable salt thereof, at least one compound of formulae I, I-a, I-b, I-c, I-d, I-e, I-f, I-g, I-h, I-i, I-j, I-k, or I-l, or a pharmaceutically acceptable salt thereof, and at least one pharmaceutically acceptable carrier. In some embodiments, the composition contains the compound of formula II or pharmaceutically acceptable salt thereof in an amount of about 1 weight percent to about 99 weight percent, where the percentages are based on the free base of said compound and on the total weight of the composition. In other embodiments, the composition contains no more than about 2.0 area percent HPLC of total organic impurities or, in other embodiments, no more than about 1.5, 1.25, 1, 0.75, 0.5, 0.25, 0.2, 0.1, 0.01, 0.005, or 0.001 area percent HPLC total organic impurities relative to the total area of the HPLC chromatogram.

In some embodiments, the composition comprises a compound of formula II or pharmaceutically acceptable salt thereof and a compound of formulae I, I-a, I-b, I-c, I-d, I-e, I-f, I-g, I-h, I-i, I-j, I-k, or I-l, or pharmaceutically acceptable salt thereof, wherein the compound of formula II or pharmaceutically acceptable salt thereof comprises about 98% and the compound of formulae I, I-a, I-b, I-c, I-d, I-e, I-f, I-g, I-h, I-i, I-j, I-k, or I-l, or pharmaceutically acceptable salt thereof comprises about 2% of the total weight of the compounds or pharmaceutically acceptable salts thereof taken together or of the total HPLC peak area of the compounds or pharmaceutically acceptable salts thereof taken together. In some embodiments, the composition comprises a compound of formula II or pharmaceutically acceptable salt thereof and a compound of formulae I, I-a, I-b, I-c, I-d, I-e, I-f, I-g, I-h, I-i, I-j, I-k, or I-l, or pharmaceutically acceptable salt thereof, wherein the compound of formula II or pharmaceutically acceptable salt thereof comprises about 99%, 99.5%, 99.6%, 99.7%, 99.8%, 99.9%, 99.95%, 99.99%, or 99.999%, and the compound of formulae I, I-a, I-b, I-c, I-d, I-e, I-f, I-g, I-h, I-i, I-j, I-k, or I-l, or pharmaceutically acceptable salt thereof comprises about 1%, 0.5%, 0.4%, 0.3%, 0.2%, 0.1%, 0.05%, 0.01%, or 0.001%, of the total weight of the compounds or pharmaceutically acceptable salts thereof taken together or of the total HPLC peak area of the compounds or pharmaceutically acceptable salts thereof taken together. In some embodiments, the compound of formulae I, I-a, I-b, I-c, I-d, I-e, I-f, I-g, I-h, I-i, I-j, I-k, or I-l, or pharmaceutically acceptable salt thereof comprises about 100 ppm, 50 ppm, 10 ppm, 1 ppm, 500 ppb, 100 ppb, or 10 ppb of the total weight of the compounds or pharmaceutically acceptable salts thereof taken together.

In some embodiments, the composition comprises a compound of formula II or pharmaceutically acceptable salt thereof and a compound of formulae I, I-a, I-b, I-c, I-d, I-e, I-f, I-g, I-h, I-i, I-j, I-k, or I-l, or pharmaceutically acceptable salt thereof, wherein the compound of formula II or pharmaceutically acceptable salt thereof comprises about 99%-99.9999%, 99.5-99.9999%, 99.6-99.9999%, 99.7-99.9999%, 99.8-99.9999%, 99.9-99.9999%, 99.95-99.9999%, 99.99-99.9999%, or 99.999-99.9999%, and the compound of formulae I, I-a, I-b, I-c, I-d, I-e, I-f, I-g, I-h, I-i, I-j, I-k, or I-l, or pharmaceutically acceptable salt thereof comprises about 10 ppm to 2%, 100 ppm to 1%, 0.0001-0.5%, 0.0001-0.4%, 0.0001-0.3%, 0.0001-0.2%, 0.0001-0.1%, 0.0001-0.05%, 0.0001-0.01%, or 0.0001-0.001% of the total weight of the compounds or pharmaceutically acceptable salts thereof taken together.

In some embodiments, the compound of formula II or pharmaceutically acceptable salt thereof and the compound of formula I, I-a, I-b, I-c, I-d, I-e, I-f, I-g, I-h, I-i, I-j, I-k, or I-l, or pharmaceutically acceptable salt thereof, are present in a ratio of about 98:2, 99:1, 99.5:0.5, 99.6:0.4, 99.7:0.3, 99.8:0.2, 99.9:0.1, 99.95:0.05, 99.99:0.01, or 99.999:0.001.

In some embodiments, the compound of any of formulae I, I-a, I-b, I-c, I-d, I-e, I-f, I-g, I-h, I-i, I-j, I-k, or I-l, or pharmaceutically acceptable salt thereof, comprises about 0.01-0.20 area percent of the HPLC chromatogram relative to the compound of formula II or pharmaceutically acceptable salt thereof. In some embodiments, the compound of formulae I, I-a, I-b, I-c, I-d, I-e, I-f, I-g, I-h, I-i, I-j, I-k, or I-l, or pharmaceutically acceptable salt thereof, comprises about 0.02-0.18, 0.03-0.16, 0.05-0.15, 0.075-0.13, 0.09-0.1, 0.1-0.2, or 0.15-0.2 area percent of the HPLC chromatogram relative to the compound of formula II or pharmaceutically acceptable salt thereof. In some embodiments, the foregoing area percentages of the HPLC chromatogram are measured relative to the total area of the HPLC chromatogram.

In some embodiments, the present invention provides any compound described above and herein in isolated form. As used herein, the term “isolated” means that a compound is provided in a form that is separated from other components that might be present in that compound's usual environment. In certain embodiments, an isolated compound is in solid form. In some embodiments, an isolated compound is at least about 50% pure as determined by a suitable HPLC method. In certain embodiments, an isolated compound is at least about 60%, 70%, 80%, 90%, 95%, 98%, 99%, 99.5%, 99.6%, 99.7%, 99.8%, 99.9%, 99.95%, 99.99%, or 99.999% as determined by a suitable HPLC method. Methods of preparation applicable to certain compounds of the invention are disclosed in US 2013/0190500, published Jul. 25, 2013, which is hereby incorporated by reference.

### 4. Uses of Compounds and Pharmaceutically Acceptable Compositions Thereof

Certain compounds described herein are found to be useful in scavenging toxic aldehydes, such as MDA and HNE. Without wishing to be bound by theory, it is believed that the compounds described herein undergo a Schiff base condensation with MDA, HNE, or other toxic aldehydes, and form a complex with the aldehydes in an energetically favorable reaction, thus reducing or eliminating aldehydes available for reaction with a protein, lipid, carbohydrate, or DNA. Importantly, compounds described herein can react with aldehydes to form a compound having a closed-ring structure that contains the aldehydes, thus trapping the aldehydes and preventing the aldehydes from being released back into the cellular milieu.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent, delay or lessen the severity of their recurrence.

The invention relates to compounds described herein for the treatment, prevention, and/or reduction of a risk of diseases, disorders, or conditions in which aldehyde toxicity is implicated in the pathogenesis.

Examples of the diseases, disorders, or conditions in which aldehyde toxicity is implicated include an ocular disease, disorder, or condition, including, but not limited to, a corneal disease (e.g., dry eye syndrome, cataracts, keratoconus, bullous and other keratopathy, and Fuch's endothelial dystrophy), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions associated with high aldehyde levels as a result of inflammation (e.g., uveitis, scleritis, ocular Stevens-Johnson Syndrome, ocular rosacea (with or without meibomian gland dysfunction)). In one example, the ocular disease, disorder, or condition is not macular degeneration, such as age-related macular degeneration (“AMD”), or Stargardt's disease. In a further example, the ocular disease, disorder, or condition is dry eye syndrome, ocular rosacea, or uveitis.

Examples of the diseases, disorders, conditions, or indications in which aldehyde toxicity is implicated also include non-ocular disorders, including psoriasis, topical (discoid) lupus, contact dermatitis, atopic dermatitis, allergic dermatitis, radiation dermatitis, acne vulgaris, Sjögren-Larsson Syndrome (SLS) and/or associated ichthyoses, neurogical and/or motor effects of SLS, SSADHD, pyridoxine-dependent epilepsy, solar elastosis/wrinkles, skin tone firmness, puffiness, eczema, smoke or irritant induced skin changes, dermal incision, a skin condition associated burn and/or wound, lupus, scleroderma, asthma, chronic obstructive pulmonary disease (COPD), rheumatoid arthritis, inflammatory bowel disease, sepsis, atherosclerosis, ischemic-reperfusion injury, Parkinson's disease, Alzheimer's disease, succinic semialdehyde dehydrogenase deficiency, multiple sclerosis, amyotrophic lateral sclerosis, diabetes, metabolic syndrome, age-related disorders, and fibrotic diseases. In a further example, the non-ocular disorder is a skin disease, disorder, or condition selected from contact dermatitis, atopic dermatitis, allergic dermatitis, and radiation dermatitis. In another example, the non-ocular disorder is a skin disease, disorder, or condition selected from Sjögren-Larsson Syndrome and/or associated ichthyoses, or a cosmetic indication associated with a burn and/or wound.

In a further example, the diseases, disorders, or conditions in which aldehyde toxicity is implicated are an age-related disorder. Examples of age-related diseases, disorders, or conditions include wrinkles, dryness, and pigmentation of the skin.

Examples of the diseases, disorders, or conditions in which aldehyde toxicity is implicated further include conditions associated with the toxic effects of blister agents or burns from alkali agents. The compounds described herein reduce or eliminate toxic aldehydes and thus treat, prevent, and/or reduce a risk of these diseases or disorders.

In some embodiments, the invention relates to the treatment, prevention, and/or reduction of a risk of an ocular disease, disorder, or condition in which aldehyde toxicity is implicated in the pathogenesis, comprising administering to a subject in need thereof a compound described herein. The ocular disease, disorder, or condition includes, but is not limited to, a corneal disease (e.g., dry eye syndrome, cataracts, keratoconus, bullous and other keratopathy, and Fuch's endothelial dystrophy in the cornea), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions where inflammation leads to high aldehyde levels (e.g., uveitis, scleritis, ocular Stevens-Johnson Syndrome, ocular rosacea (with or without meibomian gland dysfunction)). The ocular disease, disorder, or condition does not include macular degeneration, such as AMD, or Stargardt's disease. In one illustration, in the ocular disease, disorder, or condition, the amount or concentration of MDA or HNE is increased in the ocular tissues or cells. For example, the amount or concentration of aldehydes (e.g., MDA or HNE) is increased for at least 1.1-fold, 1.2-fold, 1.3-fold, 1.4-fold, 1.5-fold, 2-fold, 2.5-fold, 5-fold, 10-fold as compared to that in normal ocular tissues or cells. Compounds described herein decrease aldehyde (e.g., MDA and HNE) concentration in a concentration-dependent manner. The amount or concentration of aldehydes (e.g., MDA or HNE) can be measured by methods or techniques known in the art, such as those described in Tukozkan et al., Furat Tip Dergisi 11: 88-92 (2006).

In some embodiments, the ocular disease, disorder, or condition is dry eye syndrome. In a second class, the ocular disease, disorder, or condition is a condition associated with PRK healing and other corneal healing. For example, the invention is directed to advancing PRK healing or other corneal healing, comprising administering to a subject in need thereof a compound described herein. In a third class, the ocular disease, disorder, or condition is an ocular condition associated with high aldehyde levels as a result of inflammation (e.g., uveitis, scleritis, ocular Stevens-Johnson Syndrome, and ocular rosacea (with or without meibomian gland dysfunction). In a fourth class, the ocular disease, disorder, or condition is keratoconus, cataracts, bullous and other keratopathy, Fuchs' endothelial dystrophy, ocular cicatricial pemphigoid, or allergic conjunctivitis. The compound described herein may be administered topically or systemically, as described herein below.

In some embodiments, the invention relates to the treatment, prevention, and/or reduction of a risk of a skin disorder or condition or a cosmetic indication, in which aldehyde toxicity is implicated in the pathogenesis, comprising administering to a subject in need thereof a compound described herein. The skin disorder or condition includes, but is not limited to, psoriasis, scleroderma, topical (discoid) lupus, contact dermatitis, atopic dermatitis, allergic dermatitis, radiation dermatitis, acne vulgaris, and Sjögren-Larsson Syndrome and/or associated ichthyoses, and the cosmetic indication is solar elastosis/wrinkles, skin tone firmness, puffiness, eczema, smoke or irritant induced skin changes, dermal incision, or a skin condition associated with a burn and/or wound. In some embodiments, the disease, disorder, or condition is selected from an age-related disease, disorder, or condition of the skin, as described herein.

Various skin disorders or conditions, such as atopic dermatitis, topical (discoid) lupus, psoriasis and scleroderma, are characterized by high MDA and HNE levels (Br J Dermatol 149: 248 (2003); JEADV 26: 833 (2012); Clin Rheumatol 25: 320 (2006)). In addition, ichthyosis associated with Sjögren-Larsson Syndrome (SLS) originates from accumulation of fatty aldehydes, which disrupts the normal function and secretion of lamellar bodies (LB) and leads to intercellular lipid deposits in the strateum corneum (SC) and a defective water barrier in the skin (W. B. Rizzo et al. (2010)). In patients with SLS, mutations in the gene encoding fatty aldehyde dehydrogenase, which metabolizes fatty aldehydes, significantly reduce or ablate its activity. Thus, compounds that reduce or eliminate aldehydes, such as the compounds described herein, can be used to treat, prevent, and/or reduction of a risk of skin disorders or conditions in which aldehyde toxicity is implicated in the pathogenesis, such as those described herein. Furthermore, with an improvement to the water barrier and prevention of aldehyde-mediated inflammation (including fibrosis and elastosis (Chairpotto et al. (2005)), many cosmetic indications, such as solar elastosis/wrinkles, skin tone, firmness (puffiness), eczema, smoke or irritant induced skin changes and dermal incision cosmesis, and skin conditions associated with burn and/or wound can be treated using the method of the invention.

In some embodiments, the skin disease, disorder, or condition is psoriasis, scleroderma, topical (discoid) lupus, contact dermatitis, atopic dermatitis, allergic dermatitis, radiation dermatitis, acne vulgaris, or Sjögren-Larsson Syndrome and/or associated ichthyoses. In one exemplification, the skin disease, disorder, or condition is contact dermatitis, atopic dermatitis, allergic dermatitis, radiation dermatitis, or Sjögren-Larsson Syndrome and/or associated ichthyoses. In a second class, the cosmetic indication is solar elastosis/wrinkles, skin tone firmness, puffiness, eczema, smoke or irritant induced skin changes, dermal incision, or a skin condition associated burn and/or wound.

In some embodiments, the invention relates to the treatment, prevention, and/or reduction of a risk of a condition associated with the toxic effects of blister agents or burns from alkali agents in which aldehyde toxicity is implicated in the pathogenesis, comprising administering to a subject in need thereof a compound described herein.

Blister agents include, but are not limited to, sulfur mustard, nitrogen mustard, and phosgene oxime. Toxic or injurious effects of blister agents include pain, irritation, and/or tearing in the skin, eye, and/or mucous, and conjunctivitis and/or corneal damage to the eye. Sulfur mustard is the compound bis(2-chlorethyl) sulfide. Nitrogen mustard includes the compounds bis(2-chlorethyl)ethylamine, bis(2-chlorethyl)methylamine, and tris(2-chlorethyl)amine. Sulfur mustard or its analogs can cause an increase in oxidative stress and in particular in HNE levels, and by depleting the antioxidant defense system and thereby increasing lipid peroxidation, may induce an oxidative stress response and thus increase aldehyde levels (Jafari et al. (2010); Pal et al. (2009)). Antioxidants, such as silibinin, when applied topically, attenuate skin injury induced from exposure to sulfur mustard or its analogs, and increased activities of antioxidant enzymes may be a compensatory response to reactive oxygen species generated by the sulfur mustard (Jafari et al. (2010); Tewari-Singh et al. (2012)). Further, intervention to reduce free radical species was an effective treatment post exposure for phosgene induced lung injury (Sciuto et al. (2004)). Thus, compounds that reduce or eliminate aldehydes, such as compounds described herein, can be used to treat, prevent, and/or reduce a risk of a condition associated with the toxic effects of blister agents, such as sulfur mustard, nitrogen mustard, and phosgene oxime.

Alkali agents include, but are not limited to, lime, lye, ammonia, and drain cleaners. Compounds that reduce or eliminate aldehydes, such as compounds described herein, can be used to treat, prevent, and/or reduce a risk of a condition associated with burns from an alkali agent.

In some embodiments, the invention relates to the treatment, prevention, and/or reduction of a risk of an autoimmune, immune-mediated, inflammatory, cardiovascular, or neurological disease, disorder, or condition, or metabolic syndrome, or diabetes, in which aldehyde toxicity is implicated in the pathogenesis, comprising administering to a subject in need thereof a compound described herein. The autoimmune or immune-mediated disease, disorder, or condition includes, but is not limited to, lupus, scleroderma, asthma, chronic obstructive pulmonary disease (COPD), and rheumatoid arthritis. The inflammatory disease, disorder, or condition includes, but is not limited to, rheumatoid arthritis, inflammatory bowel disease (e.g., Crohn's disease and ulcerative colitis), sepsis, and fibrosis (e.g., renal, hepatic, pulmonary, and cardiac fibrosis). The cardiovascular disease, disorder, or condition includes, but is not limited to, atherosclerosis and ischemic-reperfusion injury. The neurological disease, disorder, or condition includes, but is not limited to, Parkinson's disease, Alzheimer's disease, succinic semialdehyde dehydrogenase deficiency (SSADHD), multiple sclerosis, amyotrophic lateral sclerosis, pyridoxine-dependent epilepsy, motor effects of SLS, and the neurological aspects of SLS (cognitive delay and spasticity). In some embodiments, a disclosed compound treats motor effects of SLS such as muscle spasticity, poor movement coordination, weakness, dysarthria, and delayed speech.

A skilled person would understand that the disease, disorder, or condition listed herein may involve more than one pathological mechanism. For example, a disease, disorder, or condition listed herein may involve dysregulation in the immunological response and inflammatory response. Thus, the above categorization of a disease, disorder, or condition is not absolute, and the disease, disorder, or condition may be considered an immunological, an inflammatory, a cardiovascular, a neurological, and/or metabolic disease, disorder, or condition.

Individuals with deficiencies in aldehyde dehydrogenase are found to have high aldehyde levels and increased risk of Parkinson's disease (PNAS 110:636 (2013)) and Alzheimer's disease (BioChem Biophys Res Commun. 273:192 (2000)). In Parkinson's disease, aldehydes specifically interfere with dopamine physiology (Free Radic Biol Med, 51: 1302 (2011); Mol Aspects Med, 24: 293 (2003); Brain Res, 1145: 150 (2007)). In addition, β-aminoadipic semialdehyde (AASA) accumulates in individuals with pyridoxine-dependent epilepsy. Furthermore, aldehydes levels are elevated in multiple sclerosis, amyotrophic lateral sclerosis, autoimmune diseases such as lupus, rheumatoid arthritis, lupus, psoriasis, scleroderma, and fibrotic diseases, and increased levels of HNE and MDA are implicated in the progression of atherosclerosis and diabetes (J. Cell. Mol. Med., 15: 1339 (2011); Arthritis Rheum 62: 2064 (2010); Clin Exp Immunol, 101: 233 (1995); Int J Rheum Dis, 14: 325 (2011); JEADV 26: 833 (2012); Clin Rheumatol 25: 320 (2006); Gut 54: 987 (2005); J Am Soc Nephrol 20: 2119 (2009)). MDA is further implicated in the increased formation of foam cells leading to atherosclerosis (Leibundgut et al., Current Opinion in Pharmacology 13: 168 (2013)). Also, aldehyde-related toxicity plays an important role in the pathogenesis of many inflammatory lung diseases, such as asthma and chronic obstructive pulmonary disease (COPD) (Bartoli et al., Mediators of Inflammation 2011, Article 891752). Thus, compounds that reduce or eliminate aldehydes, such as compounds described herein, can be used to treat, prevent, and/or reduce a risk of an autoimmune, immune-mediated, inflammatory, cardiovascular, or neurological disease, disorder, or condition, or metabolic syndrome, or diabetes. For example, compounds described herein prevent aldehyde-mediated cell death in neurons. Further, compounds described herein downregulate a broad spectrum of pro-inflammatory cytokines and/or upregulate anti-inflammatory cytokines, which indicates that compounds described herein are useful in treating inflammatory diseases, such as multiple sclerosis and amyotrophic lateral sclerosis.

As discussed above, a disclosed composition may be administered to a subject in order to treat or prevent macular degeneration and other forms of retinal disease whose etiology involves the accumulation of A2E and/or lipofuscin. Other diseases, disorders, or conditions characterized by the accumulation of A2E may be similarly treated.

In one embodiment, a compound is administered to a subject that reduces the formation of A2E. For example, the compound may compete with PE for reaction with trans-RAL, thereby reducing the amount of A2E formed. In another embodiment, a compound is administered to a subject that prevents the accumulation of A2E. For example, the compound competes so successfully with PE for reaction with trans-RAL, no A2E is formed.

Individuals to be treated fall into three groups: (1) those who are clinically diagnosed with macular degeneration or other forms of retinal disease whose etiology involves the accumulation of A2E and/or lipofuscin on the basis of visual deficits (including but not limited to dark adaptation, contrast sensitivity and acuity) as determined by visual examination and/or electroretinography, and/or retinal health as indicated by fundoscopic examination of retinal and RPE tissue for drusen accumulations, tissue atrophy and/or lipofuscin fluorescence; (2) those who are pre-symptomatic for macular degenerative disease but thought to be at risk based on abnormal results in any or all of the same measures; and (3) those who are pre-symptomatic but thought to be at risk genetically based on family history of macular degenerative disease and/or genotyping results showing one or more alleles or polymorphisms associated with the disease. The compositions are administered topically or systemically at one or more times per month, week or day. Dosages may be selected to avoid side effects, if any, on visual performance in dark adaptation. Treatment is continued for a period of at least one, three, six, or twelve or more months. Patients may be tested at one, three, six, or twelve months or longer intervals to assess safety and efficacy. Efficacy is measured by examination of visual performance and retinal health as described above.

In one embodiment, a subject is diagnosed as having symptoms of macular degeneration, and then a disclosed compound is administered. In another embodiment, a subject may be identified as being at risk for developing macular degeneration (risk factors include a history of smoking, age, female gender, and family history), and then a disclosed compound is administered. In another embodiment, a subject may have dry AMD in both eye, and then a disclosed compound is administered. In another embodiment, a subject may have wet AMD in one eye but dry AMD in the other eye, and then a disclosed compound is administered. In yet another embodiment, a subject may be diagnosed as having Stargardt disease and then a disclosed compound is administered. In another embodiment, a subject is diagnosed as having symptoms of other forms of retinal disease whose etiology involves the accumulation of A2E and/or lipofuscin, and then the compound is administered. In another embodiment a subject may be identified as being at risk for developing other forms of retinal disease whose etiology involves the accumulation of A2E and/or lipofuscin, and then the disclosed compound is administered. In some embodiments, a compound is administered prophylactically. In some embodiments, a subject has been diagnosed as having the disease before retinal damage is apparent. For example, a subject is found to carry a gene mutation for ABCA4 and is diagnosed as being at risk for Stargardt disease before any ophthalmologic signs are manifest, or a subject is found to have early macular changes indicative of macular degeneration before the subject is aware of any effect on vision. In some embodiments, a human subject may know that he or she is in need of the macular generation treatment or prevention.

In some embodiments, a subject may be monitored for the extent of macular degeneration. A subject may be monitored in a variety of ways, such as by eye examination, dilated eye examination, fundoscopic examination, visual acuity test, and/or biopsy. Monitoring can be performed at a variety of times. For example, a subject may be monitored after a compound is administered. The monitoring can occur, for example, one day, one week, two weeks, one month, two months, six months, one year, two years, five years, or any other time period after the first administration of a compound. A subject can be repeatedly monitored. In some embodiments, the dose of a compound may be altered in response to monitoring.

In some embodiments, the disclosed methods may be combined with other methods for treating or preventing macular degeneration or other forms of retinal disease whose etiology involves the accumulation of A2E and/or lipofuscin, such as photodynamic therapy. For example, a patient may be treated with more than one therapy for one or more diseases or disorders. For example, a patient may have one eye afflicted with dry form AMD, which is treated with a compound of the invention, and the other eye afflicted with wet form AMD which is treated with, e.g., photodynamic therapy.

In some embodiments, a compound for treating or preventing macular degeneration or other forms of retinal disease whose etiology involves the accumulation of A2E and/or lipofuscin may be administered chronically. The compound may be administered daily, more than once daily, twice a week, three times a week, weekly, biweekly, monthly, bimonthly, semiannually, annually, and/or biannually.

Sphingosine-1-phosphate, a bioactive signaling molecule with diverse cellular functions, is irreversibly degraded by the endoplasmic reticulum enzyme sphingosine-1-phosphate lyase, generating trans-2-hexadecenal and phosphoethanolamine. It has been demonstrated that trans-2-hexadecenal causes cytoskeletal reorganization, detachment, and apoptosis in multiple cell types via a JNK-dependent pathway. See Biochem Biophys Res Commun. 2012 Jul. 20; 424(1):18-21. These findings and the known chemistry of related α,β-unsaturated aldehydes raise the possibility that trans-2-hexadecenal interact with additional cellular components. It was shown that it reacts readily with deoxyguanosine and DNA to produce the diastereomeric cyclic 1,N(2)-deoxyguanosine adducts 3-(2-deoxy-β-d-erythro-pentofuranosyl)-5,6,7,8-tetrahydro-8R-hydroxy-6R-tridecylpyrimido[1,2-a]purine-10(3H)one and 3-(2-deoxy-β-d-erythro-pentofuranosyl)-5,6,7,8-tetrahydro-8 S-hydroxy-6 S-tridecylpyrimido[1,2-a]purine-10(3H)one. These findings demonstrate that trans-2-hexadecenal produced endogenously by sphingosine-1-phosphate lyase react directly with DNA forming aldehyde-derived DNA adducts with potentially mutagenic consequences.

Succinic semialdehyde dehydrogenase deficiency (SSADHD), also known as 4-hydroxybutyric aciduria or gamma-hydroxybutyric aciduria, is the most prevalent autosomal-recessively inherited disorder of GABA metabolism (Kim et al., 2011). It manifests a phenotype of developmental delay and hypotonia in early childhood, and severe expressive language impairment and obsessive-compulsive disorder in adolescence and adulthood. Epilepsy occurs in half of patients, usually as generalized tonic-clonic seizures although sometimes absence and myoclonic seizures occur (Pearl et al. 2014). Greater than two-thirds of patients manifest neuropsychiatric problems (i.e., ADHD, OCD and aggression) in adolescence and adulthood, which can be disabling. Metabolically, there is accumulation of the major inhibitory neurotransmitter GABA and gamma-hydroxybutyrate (GHB), a neuromodulatory monocarboxylic acid (Snead and Gibson 2005). In addition, several other intermediates specific to this disorder have been detected both in patients and the corresponding murine model. Vigabatrin (VGB; γ-vinyl-GABA), an irreversible inhibitor of GABA-transaminase, is a logical choice for treatment of SSADH deficiency because it blocks the conversion of GABA to GHB. Outcomes have been mixed, and in selected patients treatment has led to deterioration (Good 2011; Pellock 2011; Escalera et al. 2010; Casarano et al. 2011; Matern et al. 1996; Al-Essa et al. 2000). Targeted therapy for SSADHD remains elusive and, to date, interventions are only palliative.

### 5. Pharmaceutically Acceptable Compositions

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention are administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of the invention can also be administered topically, such as directly to the eye, e.g., as an eye-drop or ophthalmic ointment. Eye drops typically comprise an effective amount of at least one compound of the invention and a carrier capable of being safely applied to an eye. For example, the eye drops are in the form of an isotonic solution, and the pH of the solution is adjusted so that there is no irritation of the eye. In many instances, the epithelial barrier interferes with penetration of molecules into the eye. Thus, most currently used ophthalmic drugs are supplemented with some form of penetration enhancer. These penetration enhancers work by loosening the tight junctions of the most superior epithelial cells (Burstein, 1985, Trans Ophthalmol Soc U K 104(Pt 4): 402-9; Ashton et al., 1991, J Pharmacol Exp Ther 259(2): 719-24; Green et al., 1971, Am J Ophthalmol 72(5): 897-905). The most commonly used penetration enhancer is benzalkonium chloride (Tang et al., 1994, J Pharm Sci 83(1): 85-90; Burstein et al., 1980, Invest Ophthalmol Vis Sci 19(3): 308-13), which also works as preservative against microbial contamination. It is typically added to a final concentration of 0.01-0.05%.

In certain embodiments, the present invention is directed to a composition, as described herein, comprising a prodrug of a compound of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1

### General Reaction Sequence for Compounds of Formula I

Aldehyde trapping agents according to the present invention may be prepared as described in U.S. patent application publication US 2013/0190500, published Jul. 23, 2013, which is hereby incorporated by reference, optionally with chemical functionality present at the variable positions indicated in Scheme 1, wherein the variables are as defined above and below. Exemplary methods are described further below. Such methods may be adapted according to methods known in the art for preparation of the exemplary and other compounds of the invention.

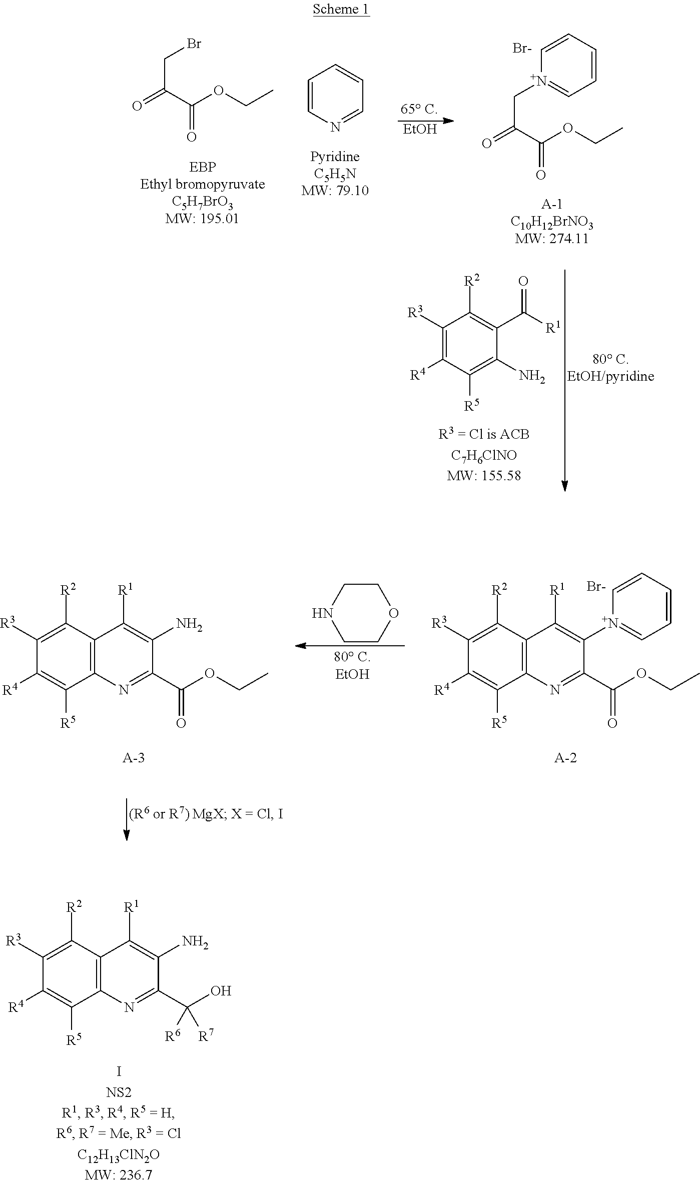

### Example 2

### Synthesis of A-1

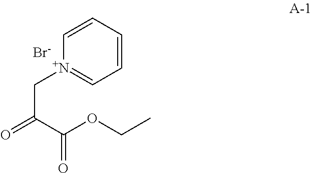

1-(3-ethoxy-2,3-dioxopropyl)pyridin-1-ium bromide. To a 2 L round bottom flask was charged ethanol (220 mL) and pyridine (31 g, 392 mmol), and the resulting solution was stirred at a moderate rate of agitation under nitrogen. To this solution was added ethyl bromopyruvate (76.6g, 354 mmol) in a slow, steady stream. The reaction mixture was allowed to stir at 65±5° C. for 2 hours.

### Example 3

### Synthesis of A-2a

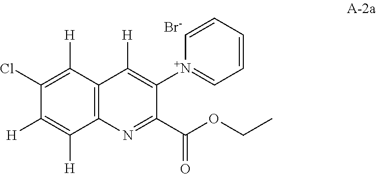 [SMILES: CCOC(=O)c1nc2ccc(Cl)cc2cc1-[n+]1ccccc1 ; [Br-]]

1-(6-chloro-2-(ethoxycarbonyl)quinolin-3-yl)pyridin-1-ium bromide. Upon completion of the 2 hour stir time in Example 2, the reaction mixture was slowly cooled to 18-22° C. The flask was vacuum-purged three times at which time 2-amino-5-chloro-benzaldehyde (ACB) (50.0 g, 321 mmol) was added directly to the reaction flask as a solid using a long plastic funnel. Pyridine (64.0 g, 809 mmol) was added followed by an EtOH rinse (10 mL) and the reaction mixture was heated at 80±3° C. under nitrogen for about 16 hours (overnight) at which time HPLC analysis indicated that the reaction was effectively complete.

### Example 4

### Synthesis of A-3a

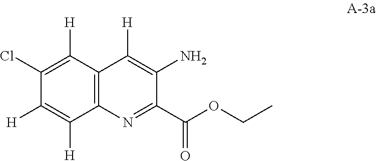 [SMILES: CCOC(=O)c1nc2ccc(Cl)cc2cc1N]

Ethyl 3-amino-6-chloroquinoline-2-carboxylate. The reaction mixture from Example 3 was cooled to about 70° C. and morpholine (76.0 g, 873 mmol)) was added to the 2 L reaction flask using an addition funnel. The reaction mixture was heated at 80±2° C. for about 2.5 hours at which time the reaction was considered complete by HPLC analysis (area% of A-3a stops increasing). The reaction mixture was cooled to 10-15° C. for the quench, work up, and isolation.

To the 2 L reaction flask was charged water (600 g) using the addition funnel over 30-60 minutes, keeping the temperature below 15° C. by adjusting the rate of addition and using a cooling bath. The reaction mixture was stirred for an additional 45 minutes at 10-15° C. then the crude A-3a was isolated by filtration using a Buchner funnel. The cake was washed with water (100 mL×4) each time allowing the water to percolate through the cake before applying a vacuum. The cake was air dried to provide crude A-3a as a nearly dry brown solid. The cake was returned to the 2 L reaction flask and heptane (350 mL) and EtOH (170 mL) were added, and the mixture heated to 70±3° C. for 30-60 minutes. The slurry was cooled to 0-5° C. and isolated by filtration under vacuum. The A-3a was dried in a vacuum drying oven under vacuum and 35±3° C. overnight (16-18 hours) to provide A-3a as a dark green solid.

### Example 5

### Synthesis of NS2

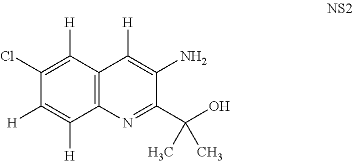 [SMILES: CC(C)(O)c1nc2ccc(Cl)cc2cc1N]

2-(3-amino-6-chloroquinolin-2-yl)propan-2-ol. To a 2 L round bottom flask was charged methylmagnesium chloride (200 mL of 3.0 M solution in THF, 600 mmol). The solution was cooled to 0-5° C. using an ice bath.

A 500 mL flask (magnetic stirring) was charged with 22.8 grams A-3a from Example 4 and THF (365 mL), stirred to dissolve, and then transferred to an addition funnel on the 2 L reaction flask. The A-3a solution was added drop-wise to the reaction flask over 5.75 hours, keeping the temperature of the reaction flask between 0-5° C. throughout the addition. At the end of the addition the contents of the flask were stirred for an additional 15 minutes at 0-5° C., then the cooling bath was removed and the reaction was allowed to stir overnight at ambient temperature.

The flask was cooled in an ice bath and the reaction mixture was carefully quenched by adding EtOH (39.5 g, 857 mmol) drop-wise to the reaction mixture, keeping the temperature of the reaction mixture below 15° C. during the course of the addition. An aqueous solution of NH₄Cl (84.7 g NH₄Cl in 415 mL water) was then carefully added and the mixture stirred under moderate agitation for about 30 minutes then transferred to a separatory funnel to allow the layers to separate. Solids were present in the aqueous phase so HOAc (12.5 g) was added and the contents swirled gently to obtain a nearly homogeneous lower aqueous phase. The lower aqueous layer was transferred back to the 2 L reaction flask and stirred under moderate agitation with 2-methyl-tetrahydrofuran (2-MeTHF) (50 mL) for about 15 minutes. The original upper organic layer was reduced in volume to approximately 40 mL using a rotary evaporator at ≦40° C. under vacuum as needed. The phases in the separatory funnel were separated and the upper 2-MeTHF phase combined with the product residue was transferred to a 500 mL flask and vacuum distilled to an approximate volume of 25 mL. To this residue was added 2-MeTHF (50 mL) and the mixture again distilled to an approximate volume of 50 mL. The crude compound NS2 solution was diluted with 2-MeTHF (125 mL), cooled to 5-10° C., and 2 M H₂SO₄ (aq) (250 mL) was slowly added and the mixture stirred for 30 minutes as the temperature was allowed to return to ambient. Heptane (40 mL) was charged and the reaction mixture stirred for an additional 15 minutes then transferred to a separatory funnel, and the layers were allowed to separate. The lower aqueous product layer was extracted with additional heptane (35 mL), then the lower aqueous phase was transferred to a 1 L reaction flask equipped with a mechanical stirrer, and the mixture was cooled to 5-10° C. The combined organic layers were discarded. A solution of 25% NaOH (aq) was prepared (NaOH, 47 g, water, 200 mL) and slowly added to the 1 L reaction flask to bring the pH to a range of 6.5-8.5.

EtOAc (250 mL) was added and the mixture was stirred overnight. The mixture was transferred to a separatory funnel and the lower phase discarded. The upper organic layer was washed with brine (25 mL), then the upper organic product layer was reduced in volume on a rotary evaporator to obtain a obtain the crude compound NS2 as a dark oil that solidified within a few minutes. The crude compound NS2 was dissolved in EtOAc (20 mL) and filtered through a plug of silica gel (23g) eluting with 3/1 heptane/EtOAc until all compound NS2 was eluted (approximately 420 mL required) to remove most of the dark color of compound NS2. The solvent was removed in vacuo to provide 14.7 g of compound NS2 as a tan solid. Compound NS2 was taken up in EtOAc (25 mL) and eluted through a column of silica gel (72g) using a mobile phase gradient of 7/1 heptane/EtOAc to 3/1 heptane/EtOAc (1400 mL total). The solvent fractions containing compound NS2 were evaporated. Compound NS2 was diluted with EtOAc (120 mL) and stirred in a flask with Darco G-60 decolorizing carbon (4.0 g) for about 1 hour. The mixture was filtered through celite using a fitted funnel, rinsing the cake with EtOAc (3×15 mL). The combined filtrates were evaporated on a rotary evaporator and compound NS2 dissolved in heptane (160 mL)/EtOAc (16 mL) at 76° C. The homogeneous solution was slowly cooled to 0-5° C., held for 2 hours, then compound NS2 was isolated by filtration. After drying in a vacuum oven for 5 hours at 35° C. under best vacuum, compound NS2 was obtained as a white solid. HPLC purity: 100% (AUC); HPLC (using standard conditions): A-2: 7.2 minutes; A-3: 11.6 minutes.

### Preparation of ACB

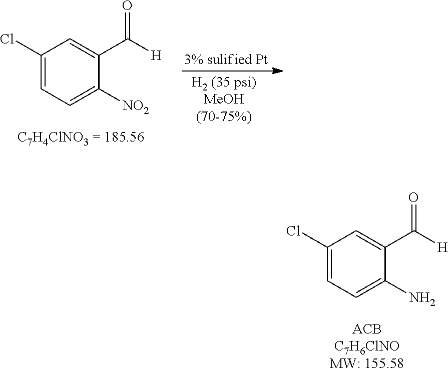 [SMILES: O=Cc1cc(Cl)ccc1[N+](=O)[O-] ; Nc1ccc(Cl)cc1C=O]

After a N₂ atmosphere had been established and a slight stream of N₂ was flowing through the vessel, platinum, sulfided, 5 wt. % on carbon, reduced, dry (9.04 g, 3.0 wt. % vs the nitro substrate) was added to a 5 L heavy walled pressure vessel equipped with a large magnetic stir-bar and a thermocouple. MeOH (1.50 L), 5-chloro-2-nitrobenzaldehyde (302.1 g, 1.63 mol), further MeOH (1.50 L) and Na₂CO₃ (2.42 g, 22.8 mmol, 0.014 equiv) were added. The flask was sealed and stirring was initiated at 450 rpm. The solution was evacuated and repressurized with N₂ (35 psi), 2×. The flask was evacuated and repressurized with H₂ to 35 psi. The temperature of the solution reached 30° C. w/in 20 min. The solution was then cooled with a water bath. Ice was added to the water bath to maintain a temperature below 35° C. Every 2h, the reaction was monitored by evacuating and repressurizing with N₂ (5 psi), 2× prior to opening. The progress of the reaction could be followed by TLC: 5-Chloro-2-nitrobenzaldehyde (R_{f}=0.60, CH₂Cl₂, UV) and the intermediates (R_{f}=0.51, CH₂Cl₂, UV and R_{f}=0.14, CH₂Cl₂, UV) were consumed to give ACB (R_{f}=0.43, CH₂Cl₂, UV). At 5 h, the reaction had gone to 98% completion (GC), and was considered complete. To a 3 L medium fitted funnel was added celite (ca. 80 g). This was settled with MeOH (ca. 200 mL) and pulled dry with vacuum. The reduced solution was transferred via cannula into the funnel while gentle vacuum was used to pull the solution through the celite plug. This was chased with MeOH (4×150 mL). The solution was transferred to a 5 L three-necked round-bottom flask. At 30° C. on a rotavap, solvent (ca. 2 L) was removed under reduced pressure. An N₂ blanket was applied. The solution was transferred to a 5 L four-necked round-bottomed flask equipped with mechanical stirring and an addition funnel. Water (2.5 L) was added dropwise into the vigorously stirring solution over 4 h. The slurry was filtered with a minimal amount of vacuum. The collected solid was washed with water (2×1.5 L), 2-propanol (160 mL) then hexanes (2×450 mL). The collected solid (a canary yellow, granular solid) was transferred to a 150×75 recrystallizing dish. The solid was then dried under reduced pressure (26-28 in Hg) at 40° C. overnight in a vacuum-oven. ACB (>99% by HPLC) was stored under a N₂ atmosphere at 5° C.

### Example 6

### In Vitro and in Vivo Assays

### LDH Cytotoxicity Assay

Primary rat cortical cultures are placed in an incubator for 24 or 48 hours and treated with various concentrations of disclosed compounds. Then 20 μL of the culture media is removed for an LDH assay as described in Bergmeyer et al., Methods of Enzymatic Analysis, 3^{rd} ed. (1983).

### ELISA Assay to Determine Amounts of Circulating Cytokines

Male C57BI/6 mice are dosed with disclosed compounds 30 minutes before they are exposed to LPS (20 mg/kg). Two hours after the LPS exposure, blood is collected from the mice and an ELISA will be conducted to determine the amounts of circulating cytokines. It is anticipated that treatment with disclosed compounds will lead to reduction in proinflammatory cytokines, such as IL-5 and IL-1β, IL-17, and TNFα. Also, treatment with disclosed compounds will result in elevation of anti-inflammatory cytokines, such as IL-10. In addition, various other chemokines, such as eotaxin, IL-12, IP-10, LIF, MCP-1, MIG, MW, and RANTES, may also be decreased by treatment with disclosed compounds.

### In Vivo Assay to Evaluate Efficacy in Treating Contact Dermatitis

To determine the efficacy of the disclosed compounds in treating contact dermatitis, phorbol myristate acetate (“PMA”) is applied topically (2.5 μg in 20 μL) to both the anterior and posterior portions of the right pinna of mice (N=10 per group). As a control, the left pinna receives 20 μL of ethanol (PMA excipient) to both the anterior and posterior portions. Six hours after the PMA application, both the right and left pinna thicknesses are determined. Measurements are determined at least twice from the same region of both ears, with care taken not to include hair or folded pinna.

### In Vivo Assay to Evaluate the Efficacy in Treating Allergic Dermatitis

To measure the efficacy of the disclosed compounds in treating allergic dermatitis, oxazolone (“OXL”) is applied (1.5%, 100 μL in acetone) to the shaved abdomens of mice. Seven days later, the thickness of the pinna of the OXL treated mice is determined. Then the disclosed compounds (100 mg/kg) or a vehicle (such as Captisol®) is administered intraperitoneally to mice followed by topical application of OXL (1%, 20 μL) 30 min later to both the anterior and posterior portions of the right pinna. As a control, the left pinna receives 20 μL of acetone (OXL excipient) to both the anterior and posterior portions. The thickness of the pinna of both ears is measured again 24 hours later. N=10 per group.

### Assay to Measure Aldehyde Trapping

To separate reaction vials is added each disclosed compound, (0.064 mmol), MDA salt (22.7% MDA, 0.064 mmol), and glyceryl trioleate (600 mg). To the mixture is added 20 wt. % Capitsol® in aqueous PBS (˜2.5 ml), followed by linoleic acid (600 mg). The reaction mixture is stirred vigorously at ambient temperature and monitored by LC/MS. It is anticipated that the disclosed compounds will quickly react with MDA to form MDA adducts.

### Schiff Base Confirmation

UV/VIS spectroscopy is used to monitor Schiff base condensation of RAL with the primary amine of a compound of the invention. The in vitro analysis of the Schiff base condensation product with RAL is performed for the disclosed compounds.

In the solution phase analysis, the λₘₐₓ value of both the free compound and the RAL Schiff base condensation product (RAL-SBC) are measured along with the value for tau of the RAL-SBC. As used herein, “RAL-SBC” means the Schiff base condensation product of RAL and a RAL-compound. Solution phase analysis is performed using a 100:1 mixture of compound and RAL using protocols known in the art. Several solvent systems were tested including aqueous, ethanol, octanol, and chloroform:methanol (various e.g., 2:1). The solution kinetics are measured and found to be highly dependent on solvent conditions.

Solid phase analysis of the Schiff base condensation is also performed using a 1:1 mixture of compound to RAL. The solid phase analysis is performed using protocols known in the art. The mixture is dried under nitrogen and condensation reaction occurs to completion.

Lipid phase analysis is performed using protocols known in the art and λₘₐₓ, tau (RAL-SBC vs. APE/A2PE), and competitive inhibition are measured. Liposome conditions are closer to in situ conditions.

### ERG Analysis of Dark Adaptation (In Vivo)

Dark adaptation is the recovery of visual sensitivity following exposure to light. Dark adaptation has multiple components including both fast (neuronal) processes and a slow (photochemical) process. Regeneration of visual pigment is related to the slow photochemical process. Night blindness results from a failure to dark adapt (loss of visual light sensitivity). It is possible to assess the potential effects of a drug on night vision by measuring dark adapted visual light sensitivity after administration of the drug.

An electroretinogram (ERG) is used to measure dark adaptation under normal vs. drug conditions. ERG is the measurement of the electric field potential emitted by retinal neurons during their response to an experimentally defined light stimulus. More specifically, ERG measures retinal field potentials at the cornea after a flash of light (e.g., 50 ms). Field strengths are 102 to 103 microvolts, originating in retinal cells.

ERG is a non-invasive measurement which can be performed on either living subjects (human or animal) or a hemisected eye in solution that has been removed surgically from a living animal. ERG requires general anesthesia which slows dark adaptation and must be factored into experimental design.

In a typical ERG analysis of dark adaptation experiment, every rat is dark-adapted for hours to reach a consistent state of light sensitivity. The rat is then “photo-bleached,” i.e., exposed briefly to light strong enough to transiently deplete the retina of free 11-cis-RAL (e.g., 2 min at 300 lux). The rat is then returned to dark immediately to initiate dark adaptation, i.e., recovery of light sensitivity due to regeneration of visual pigment. ERG is used to measure how quickly the rat adapts to dark and recovers light sensitivity. Specifically, a criterion response variable is defined for light sensitivity.

The ERG measurement is taken after a specific duration of post-bleach dark recovery (e.g., 30 min) determined previously by kinetic analysis. A curve fit is used to calculate value for the sensitivity variable and shows recovery with anesthesia in the same rat including dark adaptation kinetics for Y₅₀ and σ. Slower adaptation is observed with less light sensitivity where Y₅₀ reaches −4.0 and tau=22.6 min. Faster adaptation is observed with more light sensitivity where Y₅₀ reaches −5.5 and tau=9.2 min.

The same paradigm as described above is followed for dose ranging. In the ERG dose ranging protocol, compounds administered intraperitoneally lower light sensitivity of dark-adapted rats in a dose-dependent manner. The effect on vision decreases after 3 hours.

### NMR Analysis of RAL Reaction

NMR spectroscopy is used to monitor Schiff base condensation and ring formation of RAL with the primary amine of a compound of the invention.

### Inhibition of A2E Formation

The ability of NS2 to reduce formation of a toxic ocular aldehyde metabolite (A2E) was tested in an in vivo model of macular degeneration. abcr^{-/-} knockout mice do not express functional ABCA4, which is an ATP-binding cassette protein that transports the toxic all-trans-retinal (RAL) metabolite, A2E, out of the disc lumen to the cytoplasmic side of the disk, where the RAL can be converted to all-trans-retinol by all-trans-retinal dehydrogenase. This experiment is designed to establish proof of concept that chronic intraperitoneal administration of a RAL-trap compound lowers the accumulation rate of A2E in B6:129SvEv-Abcr (abcr−/−) mice.

### Materials and Methods:

The study was performed with B6:129SvEv-Abcr (abcr−/−) mice. Treatment groups included 24 mice (males and females) per treatment condition. Each animal was treated with one of the following conditions:
Control: a commercially available compound known clinically to modulate retinal function in humans and known experimentally to form a Schiff base adduct with free RAL, both in vitro and in vivo in animal models.
Vehicle
Compound
Untreated

The disclosed compounds will be tested at 10 mg/kg. Treatment with compounds will be administered daily for 8 weeks by intraperitoneal injection.

abcr^{-/-} knockout mice received NS2 (10 mg/kg, IP) or vehicle control (20% SBECD) daily for 56 days. A third group of animals served as untreated controls and were sacrificed on Day 1 of the study. Daily IP administration of 10 mg/kg NS2 for 56 days reduced formation of A2E by 71% (p=0.011) compared with vehicle-treated controls (data not shown). Both NS2 and vehicle were well tolerated.

The results imply that NS2 was able to diminish formation of A2E by trapping RAL, suggesting that NS2 is effective in treating retinal diseases in which aldehydes play a role.

### Chemistry:

The experiments used a variety of chemistry services. For example, these experiments use commercially available compounds with analytical specification sheets to characterize the impurities. Compounds were also synthesized. Formulations of the compound were suitable for intraperitoneal (i.p.) injection.

### Biology and Biochemistry:

The experiments described herein used a variety of biology and biochemistry services. If necessary, non-toxic doses of compounds of the invention, formulated for treatment with an eye drop or other formations, may be established, e.g., in the rabbit with an ocular irritation protocol. Alternatively, if initial in cello analysis shows cytotoxicity, compound doses are reduced to avoid exposure to cytotoxic amounts. Light responses were characterized by ERG (Weng, et al., Cell 98:13, 1999). Intracellular A2E concentration of retinal RPE cell extracts were measured using an analytical method such as those described by Karan et al., 2005; Radu et al., 2003; and Parish et al., PNAS 95:14609, 1998.

Morphology of retinal and RPE tissue is assessed with light microscopy histology techniques (Karan et al. 2005, with the exception that electron microscopy is not used in the experiments described herein).

### Example 7

### Evaluation of Dose Responses for Protective Activity from Hydrogen Peroxide Toxicity in Dissociated Hippocampal Cultures

Test Agents

Test agents were purchased from commercial suppliers or prepared as described herein and using methods known in the art.

Formulations and Stock Solution Preparation

Test agents were prepared in two formulations: dimethyl sulfoxide (DMSO) or Captisol®.

Culture Conditions Designed to Detect Test Compound-Mediated Neuroprotection from Oxidative Stress Associated with Hydrogen Peroxide

Rat hippocampal cultures are prepared as previously described (Brenneman D E, Smith G R, Zhang Y, Du Y, Kondaveeti S K, Zdilla M J, Reitz A B. (2012) J. Molecular Neuroscience, 47:368-379). Under these conditions, the cultures are at least 90% neuronal. The most abundant non-neuronal cells are astrocytes.

Cultures were plated at a density of 10,000 cells per well, in 96-well plates. Cultures are treated between day 10 and day 21 after dissociation of E18 hippocampal tissue. Hydrogen peroxide was added to the cultures about 10 minutes after treatment with test compound or cannabidiol (CBD) control compound. There were five replicates per treatment condition.

Cultures were plated in B27/Neural Basal. On the day of treatment, all cultures were given a complete change of medium into B27/Neural Basal Medium without antioxidants.

As previously determined (Brenneman et al., 2012), 10 μM hydrogen peroxide is used to produce toxicity and oxidative stress. This concentration of hydrogen peroxide has been observed in the hippocampus of rats in a kainite-induced model of status epilepticus [Jarrett, S G, Liang, L-P, Hellier, J L, Staley, K J and Patel, M. (2008) Neurobiol. Dis 30(1): 130-138].

The positive control used in all studies was 10 μM cannabidiol (CBD), which has been shown to protect against oxidative stress in primary neurons [Brenneman, D E, Petkanas, D and Kinney, W. A. (2014) Annual Symposium on the Cannabinoids, page 129].

Neither the negative control wells, the hydrogen peroxide wells, nor the positive control wells contained any drug vehicle.

Assays

Both assays were conducted simulateously in the same wells.

1. Neuronal viability assay: CFDA (carboxyfluorescein diacetate) dye is taken up by all live cells and cleaved by esterases in the inner leaflet of the plasma membrane, releasing fluorescein into the cytosol. Live neurons cannot extrude this dye, whereas efflux of the dye from non-neuronal cells can occur over time, thus the assay specifically detects only neurons. Cultures are read in a fluorimeter and intracellular dye intensity is proportional to the live neuronal population. Original reference: Petroski, R E and Geller H M. (1994) Selective labeling of embryonic neurons cultures on astrocyte monolayers with 5(6)-carboxyfluorescein diacetate (CFDA) J. Neurosci. Methods 52:23.32.

2. Cell death assay: Propidium iodide is excluded from live cells, but can access dead cells and bind to DNA. The assay detects both necrotic and apoptotic cell death; it does not distinguish between neuronal cell death and non-neuronal cell death. See Sarafian T A, Kouyoumjian S, Tashkin D, Roth M D. (2002) Tox. Letters. 133: 171-179.

3. Data Analyses
a. EC₅₀ values (concentration of ligand that produced 50% of maximal effective response) were calculated for each of these tested compounds.
b. All data were statistically analyzed by an Analysis of Variance with the Multiple Comparisons versus Control Group (Holm-Sidak) method. Statistical significance was taken at the P<0.05 level. In all cases, comparisons were made to the negative control (10 μM hydrogen peroxide treatment).

### Example 8

### Assay Results for Aldehyde Adduct Formation, 4-HNE Consumption, and Equilibration Over Time

Five compounds were examined:

2-(3-aminoquinolin-2-yl)propan-2-ol (I-1)

2-(3-amino-5-chloroquinolin-2yl)propan-2-ol (I-2)

2-(3-amino-7-chloroquinolin-2-yl)propan-2-ol (I-3)

2-(3-amino-8-chloroquinolin-2-yl)propan-2-ol (I-4)

2-(3-amino-6-bromoquinolin-2-yl)propan-2-ol (I-5)

NS2 was also examined for comparison.

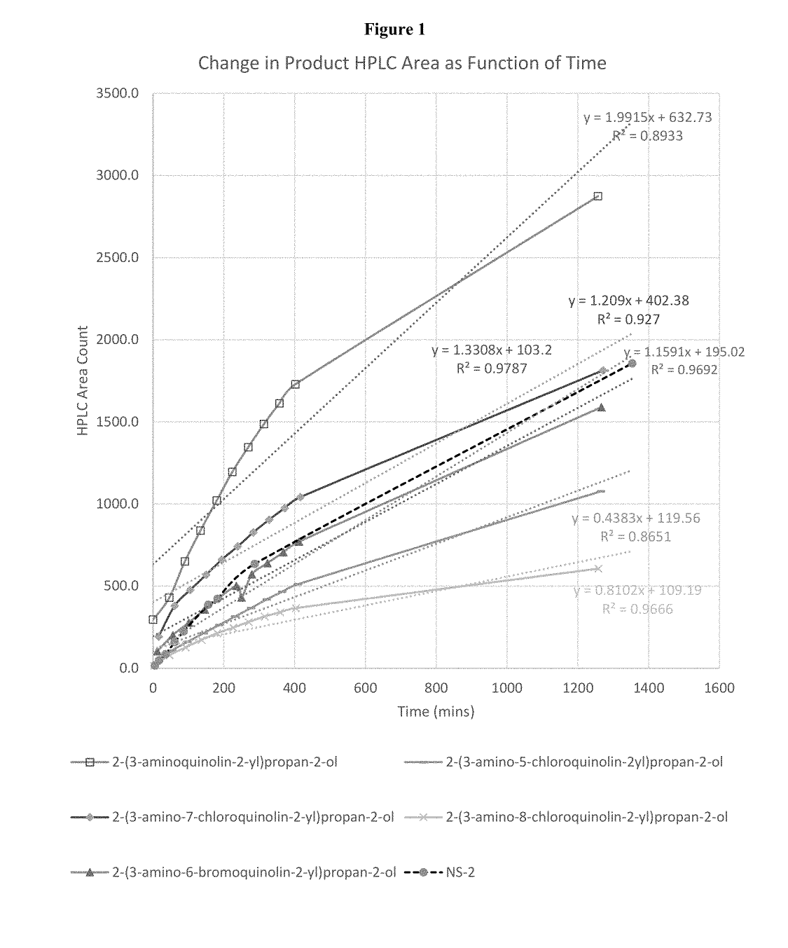

FIG. 1 shows rates of formation of aldehyde adducts over a 23 h time period for NS2 and the exemplary compounds I-1, I-2, I-3, I-4, and I-5. It was found that all samples bind (positive increase in product HPLC peak over time), although one binds less well than the others. It is not possible to conclude if this is the result of poor dissociation (from cyclodextrin) or poor interaction with the aldehyde. Best fit lines over this period give excellent fit to data. Rate of product peak increase can be used as an approximation of binding kinetics; however, it does not provide any way to separate kinetics of dissociation (from cyclodextrin) and kinetics of binding. It can be used to relatively rank each of the samples examined, including NS2. The data were first evaluated over a 7 h time window. This resulted in the following rankings from most effective to least:

| | |
|---|---|
| 1. 2-(3-aminoquinolin-2-yl)propan-2-ol | (Gradient 3.68, R.Sq. 0.993) |
| 2. NS2 | (Gradient 2.22, R.Sq. 0.996) |
| 3. 2-(3-amino-7-chloroquinolin-2-yl)- | (Gradient 2.02, R.Sq. 0.984) |
| propan-2-ol | |
| 4. 2-(3-amino-6-bromoquinolin-2-yl)- | (Gradient 1.63, R.Sq. 0.983) |
| propan-2-ol | |
| 5. 2-(3-amino-8-chloroquinolin-2-yl)- | (Gradient 1.18, R.Sq. 0.997) |
| propan-2-ol | |
| 6. 2-(3-amino-5-chloroquinolin-2y1)- | (Gradient 0.86, R.Sq. 0.983) |
| propan-2-ol | |

Similar results were obtained when the window was extended to 23 h. However, two of the compounds yielded lower R. Sq. values in this context.

| | |
|---|---|
| 1. 2-(3-aminoquinolin-2-yl)propan-2-ol | (Gradient 1.99, R.Sq. 0.893) |
| 2. NS2 | (Gradient 1.33, R.Sq. 0.979) |
| 3. 2-(3-amino-7-chloroquinolin-2-yl)- | (Gradient 1.21, R.Sq. 0.927) |
| propan-2-ol | |
| 4. 2-(3-amino-6-bromoquinolin-2-yl)- | (Gradient 1.16, R.Sq. 0.969) |
| propan-2-ol | |
| 5. 2-(3-amino-8-chloroquinolin-2-yl)- | (Gradient 0.81, R.Sq. 0.967) |
| propan-2-ol | |
| 6. 2-(3-amino-5-chloroquinolin-2yl)- | (Gradient 0.44, R.Sq. 0.967) |
| propan-2-ol | |

One possible explanation is that the two kinetic components (dissociation and binding) are no longer balanced and one is the determining factor. A follow-up experiment would be to closely track one sample over 60-70 injections to establish where the slope change occurs (this would potentially give access point to separate dissociation and binding kinetic components).

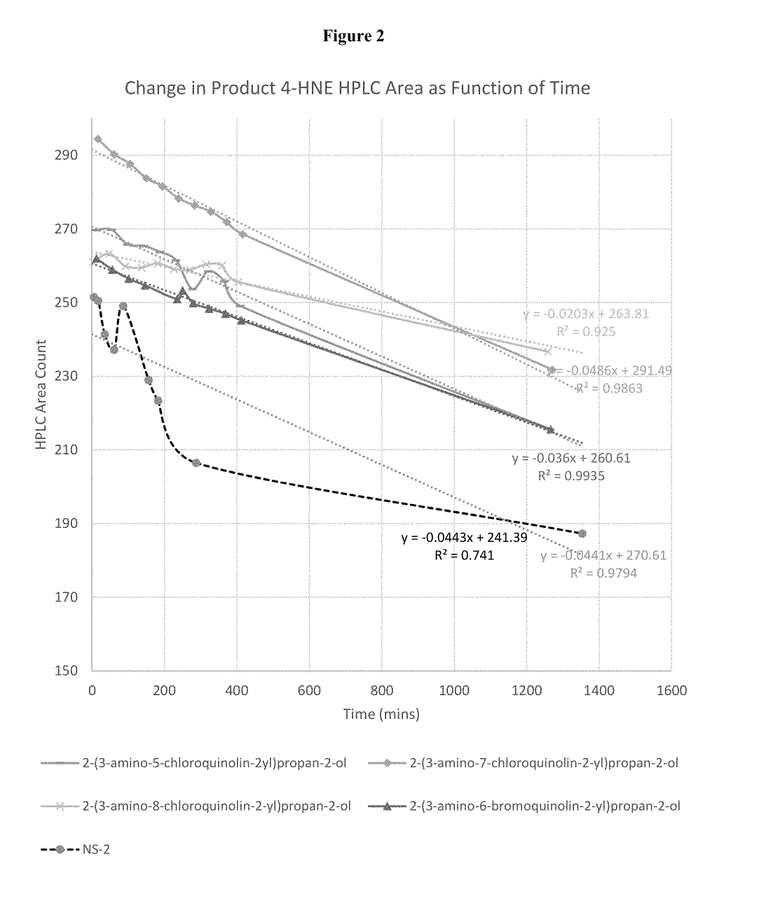

FIG. 2 shows consumption of 4-HNE over time (23-hour formation period) for NS2 and the exemplary compounds. Five of 6 samples show consumption of 4-HNE. One sample (2-(3-aminoquinolin-2-yl)propan-2-ol) overlaps the 4-HNE HPLC peak using the current method. Best fit lines over this period give poorer fit to data than product formation data. Rate of 4-HNE consumption can be used as an approximation of binding kinetics. As before, the data do not provide any way to separate kinetics of dissociation (from cyclodextrin) and kinetics of binding. The data were used to rank relatively each of the samples examined, including NS2 but excluding 2-(3-aminoquinolin-2-yl)propan-2-ol. During the first 7 h, the data yielded the following rankings from most effective to least (analysis at 254 nm):

| | |
|---|---|
| 1. NS2 | (Gradient −0.15, R.Sq. 0.903) |
| 2. 2-(3-amino-7-chloroquinolin-2-yl)- | (Gradient −0.06, R.Sq. 0.991) |
| propan-2-ol | |
| 3. 2-(3-amino-5-chloroquinolin-2yl)- | (Gradient −0.05, R.Sq. 0.898) |
| propan-2-ol | |
| 4. 2-(3-amino-6-bromoquinolin-2-yl)- | (Gradient −0.04, R.Sq. 0.971) |
| propan-2-ol | |
| 5. 2-(3-amino-8-chloroquinolin-2-yl)- | (Gradient −0.01, R.Sq. 0.461) |
| propan-2-ol | |

Analysis at 23 h provided the following rankings from most effective to least:

| | |
|---|---|
| 1. 2-(3-amino-7-chloroquinolin-2-yl)- | (Gradient −0.05, R.Sq. 0.986) |
| propan-2-ol | |
| 2. 2-(3-amino-5-chloroquinolin-2yl)- | (Gradient −0.04, R.Sq. 0.979) |
| propan-2-ol | |
| 3. NS2 | (Gradient −0.04, R.Sq. 0.741) |
| 4. 2-(3-amino-6-bromoquinolin-2-yl)- | (Gradient −0.04, R.Sq. 0.994) |
| propan-2-ol | |
| 5. 2-(3-amino-8-chloroquinolin-2-yl)- | (Gradient −0.02, R.Sq. 0.925) |
| propan-2-ol | |

Note, differences between bold numbers are very small (Gradient numbers rounded to value shown).

The following table summarizes the above data:

TABLE 2

| | Formation | | Consumption | |
|---|---|---|---|---|
| | of Product | | of 4-HNE | |
| | | 23 | | 23 |
| Compound | 7 Hours | Hours | 7 Hours | Hours^{¶} |
| 2-(3-aminoquinolin-2-yl)propan-2- | 1 | 1 | n/a | n/a |
| ol | | | | |
| NS2 | 2 | 2 | 1 | 3 |
| 2-(3-amino-7-chloroquinolin-2- | 3 | 3 | 2 | 1 |
| yl)propan-2-ol | | | | |
| 2-(3-amino-6-bromoquinolin-2- | 4 | 4 | 4 | 4 |
| yl)propan-2-ol | | | | |
| 2-(3-amino-8-chloroquinolin-2- | 5 | 5 | 5 | 5 |
| yl)propan-2-ol | | | | |
| 2-(3-amino-5-chloroquinolin- | 6 | 6 | 3 | 2 |
| 2yl)propan-2-ol | | | | |

^{¶}Small differences between samples ranking 1-4, essentially identical

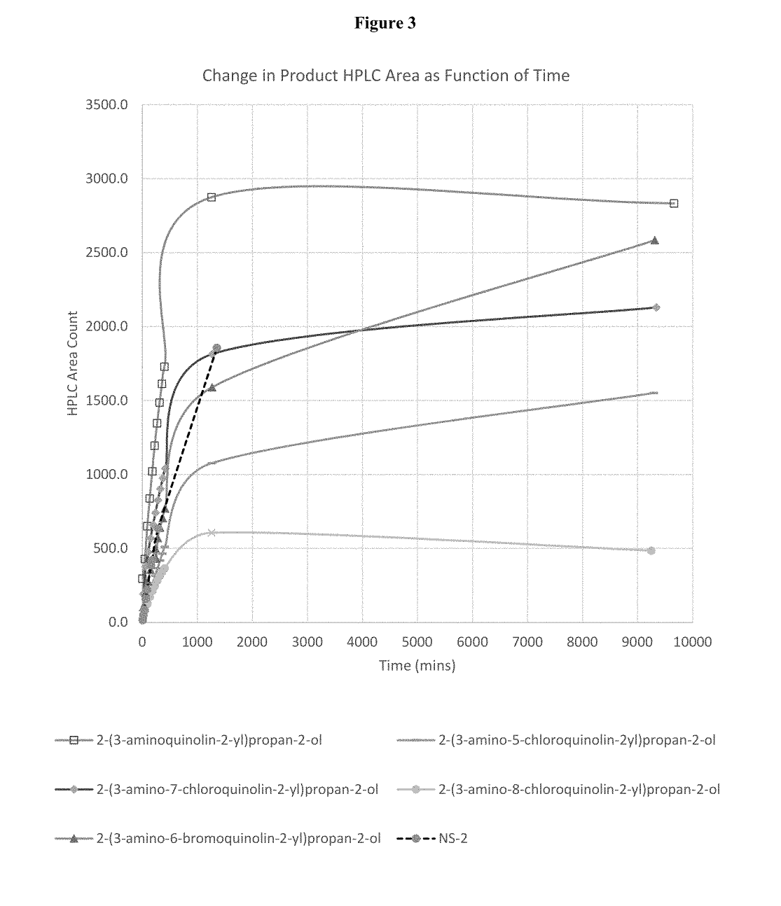

FIG. 3 shows shows rates of formation of aldehyde adducts over a one-week time period for NS2 and exemplary compounds of the present invention to measure whether compounds reached equilibrium. During this time period 3 of the 5 samples reached equilibrium.

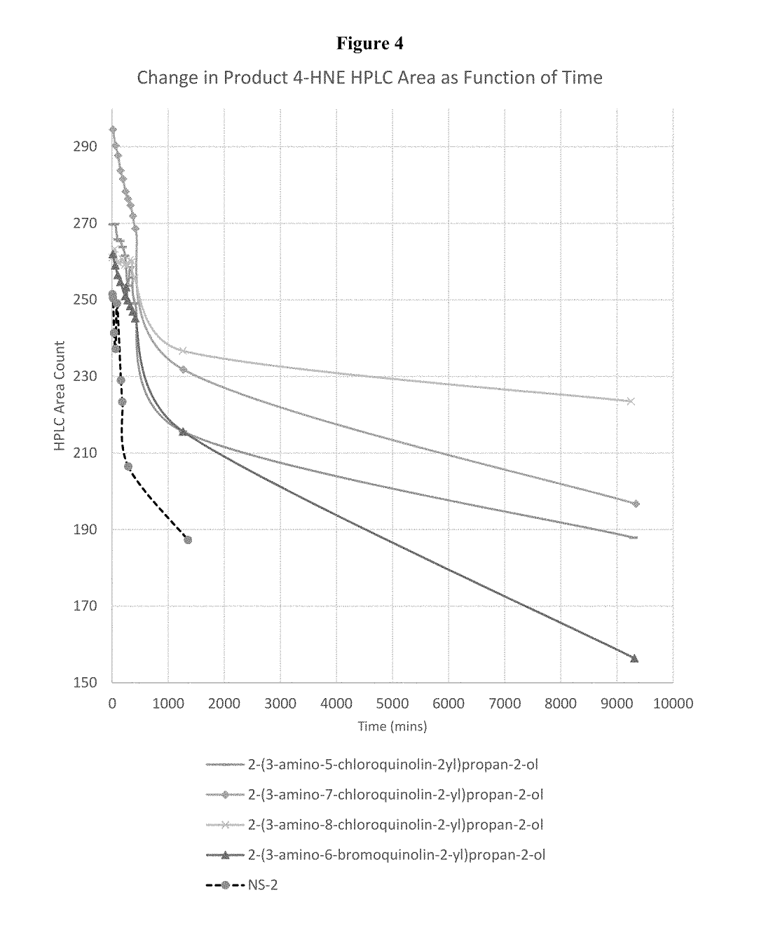

FIG. 4 shows shows consumption of 4-HNE over a one-week time period for NS2 and exemplary compounds of the present invention to measure whether compounds reached equilibrium during this time period. The samples appeared to reach equilibrium, with the ongoing decrease in HNE amounts possibly due to another degradative pathway. This is because the decrease in HNE is greater than the corresponding increase in adduct (shown in FIG. 3) for at least 2-(3-amino-8-chloroquinolin-2-yl)propan-2-ol and 2-(3-amino-7-chloroquinolin-2-yl)propan-2-ol.

### Example 9

### Ex Vivo SSADH Studies

Methods:

Three and one half (3.5) days after birth, B6.129-Aldh5a1^{tm1Kmg}/J (SSADH null) mice and wild type (wt) littermates animals were sacrificed and brains were harvested. Brains were sliced into sagittal sections of approximately 0.5 mm, and incubated in 100 μg/mL of Compound 1 (NS2), Compound 2 (I-1), or vehicle for 24 hours. Brain slices and the incubation media (sup) were then analyzed by HPLC for GHB and GABA content.

Results:

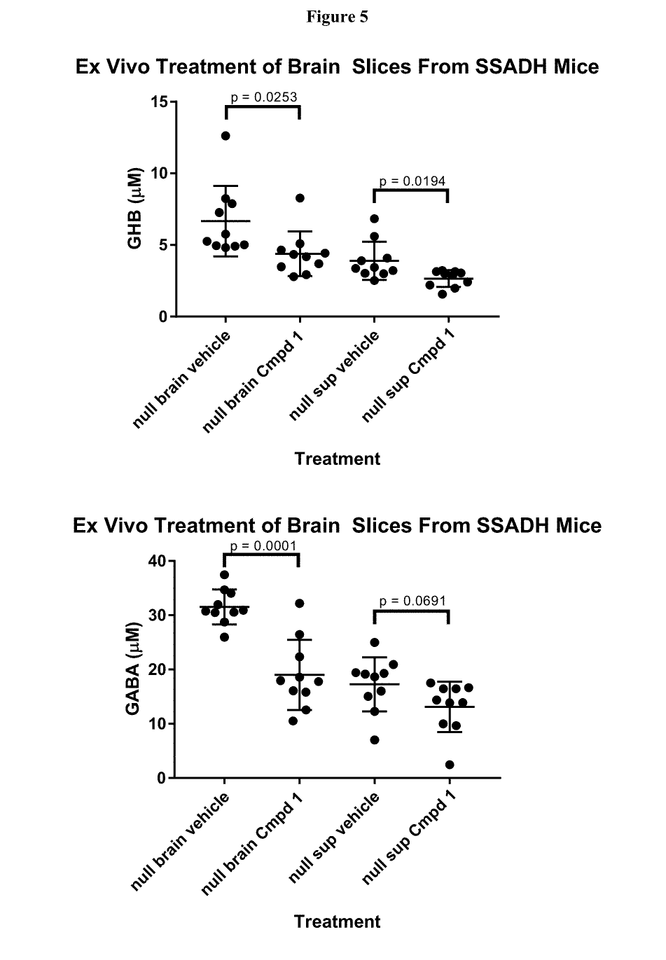

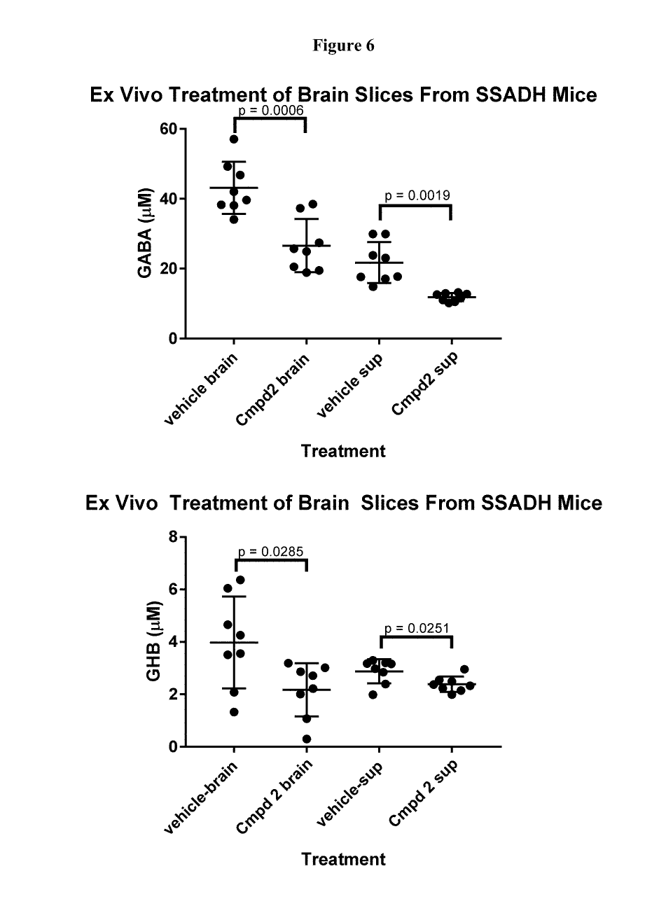

NS2 effects on measured GABA and GHB content in brain slices of the SSADH null mice are shown in FIG. 5. I-1 effects on measured GABA and GHB content in brain slices of the SSADH null mice are shown in FIG. 6. Each compound decreased GABA and GHB compared to the controls. As noted above, in SSADHD patients there is an accumulation of GABA and GHB. Accordingly, the ability of I-1 and related compounds to decrease GABA and GHB in this disease model suggests potential to treat SSADHD in humans.

### Example 10

### Formation of 4-HNE Adduct With Exemplary Compounds

The ability of exemplary compounds to react with 4-HNE and form the corresponding adduct was measured. All compounds were analyzed by HPLC at 254 nm for their purity prior to the reaction.

Reactions were carried out with one equivalent of 4-HNE and 2 equivalents of NS2 analog. Area under the curve (AUC) of each adduct was plotted over time.

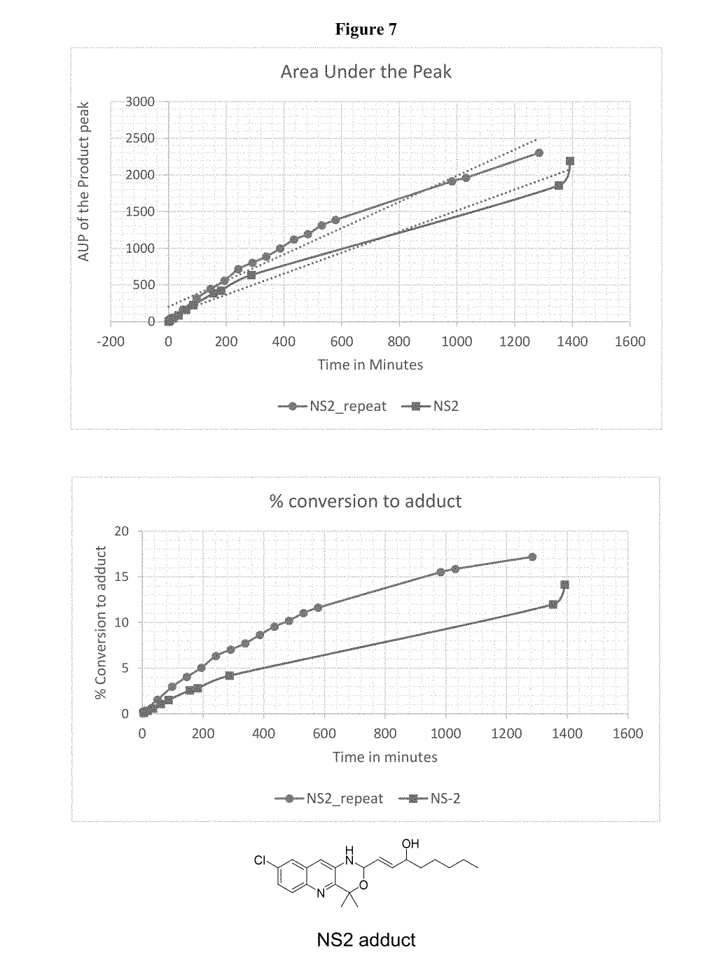

FIG. 7 shows assay results for NS2. The assay was performed twice, with the measurements on different days. NS2 formed the corresponding adduct with 4-HNE. The two results were similar to each other, and were close enough to be within the measurement error for the HPLC instrument.

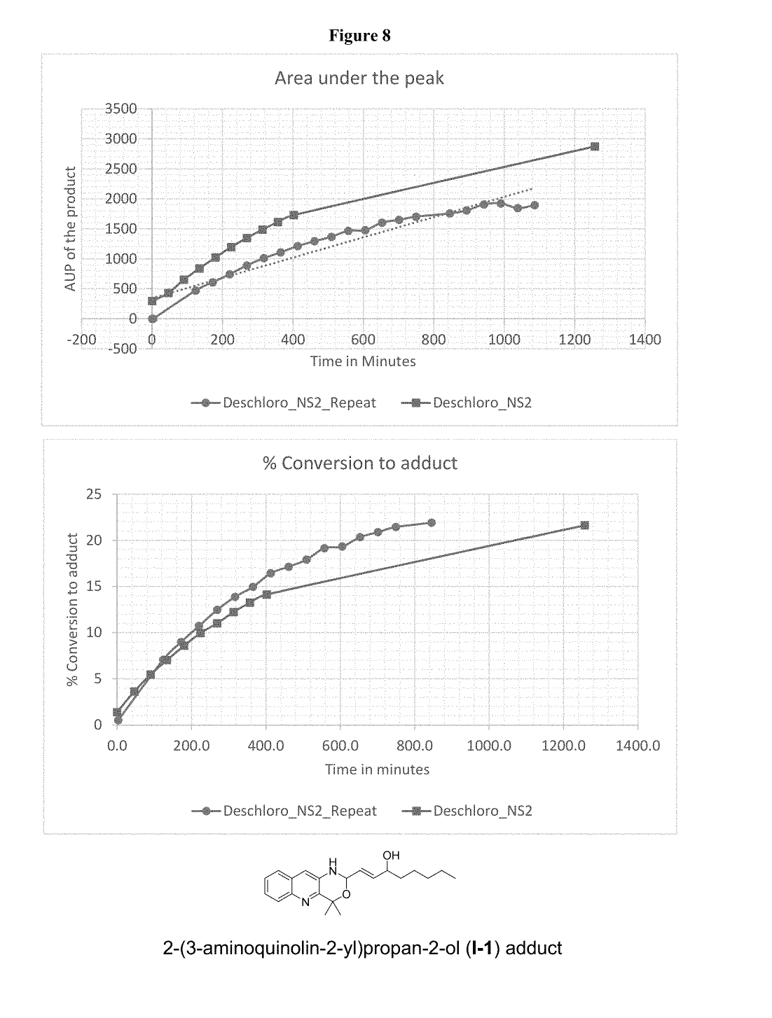

FIG. 8 shows assay results for I-1. The assay was performed twice, with the measurements on different days. I-1 formed the corresponding adduct with 4-HNE. The two results were similar to each other, and were close enough to be within the measurement error for the HPLC instrument.

We claim:

1. A compound of formula I: 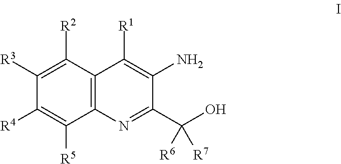
or a pharmaceutically acceptable salt thereof, wherein:
R¹ is H, D, or halogen;
R² is H, D, or halogen;
R³ is H, D, Br, or I;
R⁴ is H, D, or halogen;
R⁵ is H, D, or halogen;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms.

2. A composition comprising a compound of formula II: 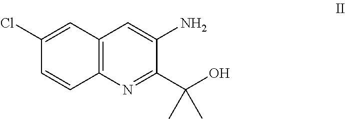 [SMILES: CC(C)(O)c1nc2ccc(Cl)cc2cc1N]
or a pharmaceutically acceptable salt thereof, and at least one compound of formula I: 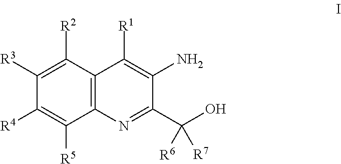
or a pharmaceutically acceptable salt thereof, wherein:
R¹ is H, D, or halogen;
R² is H, D, or halogen;
R³ is H, D, Br, or I;
R⁴ is H, D, or halogen;
R⁵ is H, D, or halogen;
R⁶ is C₁₋₄ aliphatic optionally substituted with 1, 2, or 3 deuterium or halogen atoms; and
R⁷ is C₁₋₄ aliphatic optionally substituted with 1, 2, or 3 deuterium or halogen atoms.

3. The composition of claim 2, wherein the compound of formula I is selected from the following, or a pharmaceutically acceptable salt thereof:

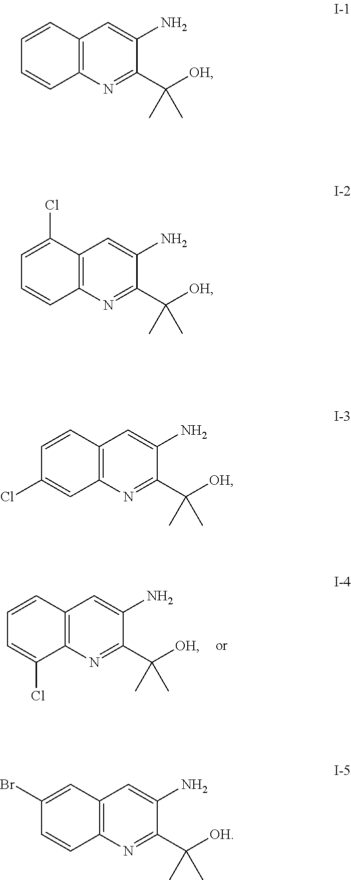 [SMILES: CC(C)(O)c1nc2ccccc2cc1N ; CC(C)(O)c1nc2cccc(Cl)c2cc1N ; CC(C)(O)c1nc2cc(Cl)ccc2cc1N ; CC(C)(O)c1nc2c(Cl)cccc2cc1N ; CC(C)(O)c1nc2ccc(Br)cc2cc1N]

4. The compound according to claim 1, wherein the compound is of formula I-a: 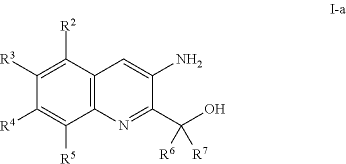
or a pharmaceutically acceptable salt thereof.

5. The compound according to claim 1, wherein the compound is of formula I-b: 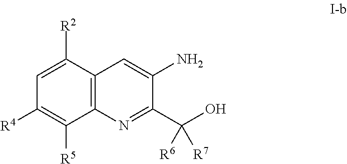
or a pharmaceutically acceptable salt thereof.

6. The compound according to claim 1, wherein the compound is of formulae I-c, I-d, I-e, or I-f: 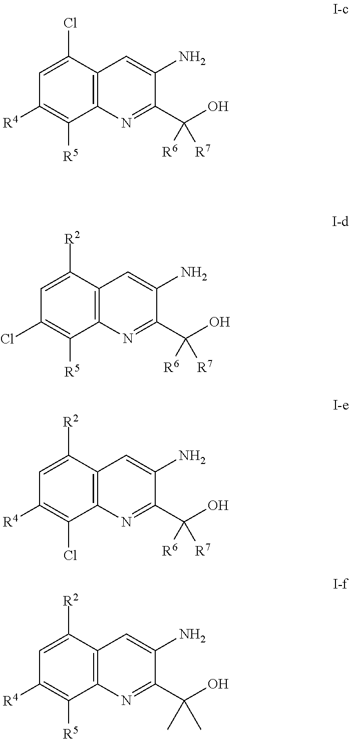
or a pharmaceutically acceptable salt thereof.

7. The compound according to claim 1, wherein the compound is of formulae I-g, I-h, I-i, or I-j: 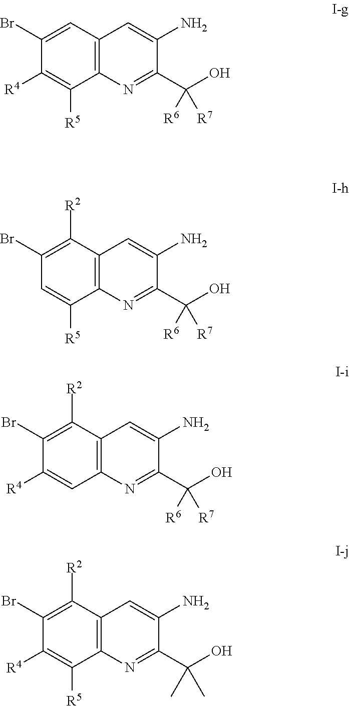
or a pharmaceutically acceptable salt thereof.

8. The compound according to claim 1, wherein the compound is of formula I-k or I-l: 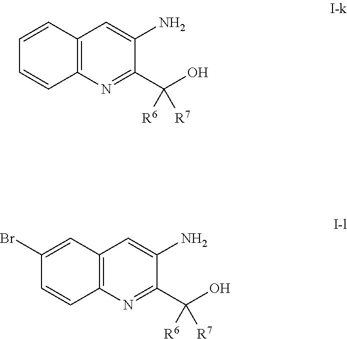
or a pharmaceutically acceptable salt thereof.

9. The compound of claim 1, wherein R¹ is H.

10. The compound of claim 9, wherein R² is H or Cl.

11. The compound of claim 1, wherein R³ is H.

12. The compound of claim 1, wherein R³ is Br.

13. The compound of claim 9, wherein R⁴ is H or Cl.

14. The compound of claim 9, wherein R⁵ is H or Cl.

15. The compound of claim 1, wherein R⁶ is C₁₋₄ alkyl.

16. The compound of claim 1, wherein R⁶ and R⁷ are methyl.

17. A compound selected from the following, or a pharmaceutically acceptable salt thereof:

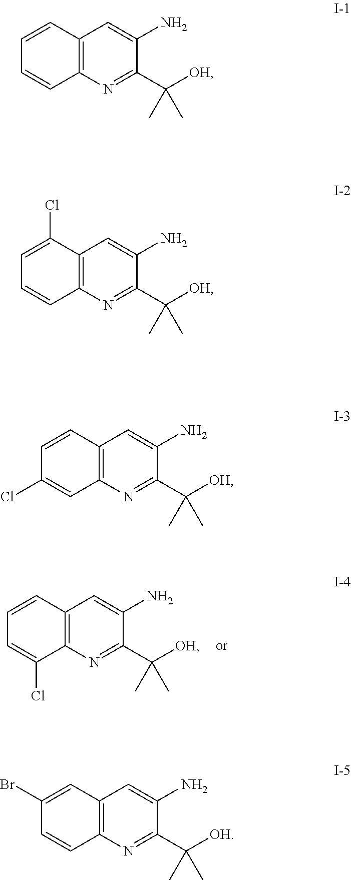 [SMILES: CC(C)(O)c1nc2ccccc2cc1N ; CC(C)(O)c1nc2cccc(Cl)c2cc1N ; CC(C)(O)c1nc2cc(Cl)ccc2cc1N ; CC(C)(O)c1nc2c(Cl)cccc2cc1N ; CC(C)(O)c1nc2ccc(Br)cc2cc1N]

18. A pharmaceutical composition comprising a compound according to claim 1 and a pharmaceutically acceptable adjuvant, carrier, or vehicle.

19. The composition according to claim 18, further comprising an additional therapeutic agent.

20. A method of treating macular degeneration or a retinal disease whose etiology involves accumulation of A2E and/or lipofuscin in a subject, comprising administering to the subject an effective amount of a compound or composition according to claim 1 or a pharmaceutically acceptable salt thereof, and thereby reducing the level of A2E accumulation relative to the level of A2E accumulation in said subject without administration of the compound or composition or pharmaceutically acceptable salt thereof.

21. A method of treating, preventing, or reducing a risk of a disease, disorder, condition, or cosmetic indication in which aldehyde toxicity is implicated in a subject in need thereof, comprising administering topically or systemically to the subject a compound or composition according to claim 1.

22-44. (canceled)
